(19) United States
(12) Patent Application Publication
(10) Pub. No.: US 2021/0162128 A1
(43) Pub. Date: Jun. 3, 2021

(54) INDIVIDUALLY TAILORED DELIVERY OF AGENTS TO OPTIMIZE EFFICACY

(71) Applicant: Xinova, LLC, Seattle, WA (US)

(72) Inventor: Michael Keoni MANION, Seattle, WA (US)

(73) Assignee: Xinova, LLC, Seattle, WA (US)

(21) Appl. No.: 17/267,102

(22) PCT Filed: Aug. 13, 2018
(86) PCT No.: PCT/US18/46407

§ 371 Date: Feb. 9, 2021

(51) Int. Cl. A61M 5/172; A61B 5/00; A61B 7/00; A61M 5/14; A61F 9/00; A61M 31/00; A61M 11/00; A61M 15/00; A61M 5/19

(52) U.S. Cl. CPC A61M 5/1723; A61B 2560/0242; A61B 5/4839; A61B 5/6804; A61B 7/00; A61M 5/1407; A61F 9/0008; A61M 31/00; A61M 11/00; A61M 15/0086; A61M 5/19; A61B 5/4088; A61B 5/4848; A61B 2562/0219; A61M 2205/3592; A61M 2205/3584; A61M 2205/52; A61B 5/681

(57) ABSTRACT

Technologies are described to control and optimize a delivery timing and dosage of agents, such as nootropics, in an individually tailored manner. Nootropics enhance cognitive functions, including executive functions, memory, creativity and/or motivation. Typically, nootropic regimes require a person to orally take multiple different agents at different dosages and at different times of the day. Embodiments are directed to instructing a delivery device to deliver an agent at a predefined time and dosage to an individual, receiving an input associated with parameters related to a physiological function and/or a cognitive function of the individual, and processing the input to analyze an efficacy of the agent. The predefined time and/or dosage for delivery of the agent to the individual may then be adjusted based on the analysis. The delivery device may be a standalone device or may be integrated with various other items.

### BACKGROUND

Unless otherwise indicated herein, the materials described in this section are not prior art to the claims in this application and are not admitted to be prior art by inclusion in this section.

Certain drug or supplement regimens may require a person to take multiple different agents at different dosages and at different times of the day. The agents may individually target specific functions that in combination effectively contribute to an overall improvement in the person's physical or mental health and/or functioning. However, these types of regimens may be very difficult for the person to self-manage. For example, if the agents are delivered via oral routes, pills may be forgotten or taken at the incorrect times, which may render the agents less effective, individually or in combination. Additionally, too many pills or pills in an incorrect combination may be taken, which may potentially lead to harmful effects.

A nootropics program is one example of a regimen that requires a person to take multiple different agents at different dosages and at different times of the day for what is often referred to as a full stack program. Nootropics are deliverable agents, such as drugs, supplements, or other similar substances, that enhance cognitive functions, including executive functions, memory, creativity and/or motivation. Thus, nootropics may assist with sleep, wakefulness, attention, motivation, energy, and creativity, among other examples. Currently, most of these agents are either in pill or powder form that are taken orally, and thus a full stack program typically requires a person to take multiple pills at different times of the day.

There are several significant limitations with the current approaches to the delivery of nootropics. For example, delivery via enteral routes may result in the agents breaking down and/or not being absorbed well in the gastrointestinal tract. Given the oral dosing, which is a bolus approach with a set dose, the pharmacokinetic/pharmacodynamic (PK/PD) is limited to these profiles, and it is impossible to deliver these agents during sleep. Additionally, compliance is dependent on the person taking the correct agents in the correct amount at the right time, which is complicated with multiple agents at different dosages and at different times of the day. There is no individual tailoring of the effectiveness of certain agents or combinations of agents for optimal effectiveness. Moreover, there is no feedback to determine how to modify the dose and/or schedule of the agents being delivered in response to certain events (e.g., exam times, extra stress, travel, illness, etc.) and over time to optimize the cognitive enhancement effectiveness. The combination of the above-discussed limitations results in substandard dosing and, consequently, effectiveness of a nootropics program.

### SUMMARY

The present disclosure generally describes techniques for controlling and optimizing delivery of agents to an individual through a delivery device in a tailored manner.

According to some examples, methods to control delivery of agents are provided. A delivery device may be instructed to deliver an agent at a predefined time and a predefined dosage to an individual. Following the delivery of the agent to the individual, an input associated with one or more parameters related to a physiological function and/or a cognitive function of the individual may be received. The input may be processed to analyze an efficacy of the agent with respect to the physiological and/or cognitive functions of the individual, and the predefined time and/or dosage for delivery of the agent may be adjusted based on the analysis.

According to other examples, computing devices configured to control delivery of agents are described. An example computing device may include a memory configured to store instructions and a processor coupled to the memory. The processor in conjunction with the instructions stored on the memory may be configured to instruct a delivery device to deliver an agent at a predefined time and a predefined dosage to an individual. Following the delivery of the agent to the individual, the processor may be configured to receive an input associated with one or more parameters related to a physiological function and/or a cognitive function of the individual. The processor may then be configured to process the input to analyze an efficacy of the agent with respect to the physiological and/or cognitive functions of the individual, and adjust the predefined time and/or the predefined dosage for delivery of the agent to the individual based on the analysis.

According to further examples, systems configured to deliver agents are described. An example system may include a delivery device configured to deliver an agent to an individual and a computing device coupled to the delivery device and configured to control the delivery of the agent by the delivery device. The delivery device may include a reservoir configured to hold the agent, a dispenser configured to receive the agent upon release from the reservoir and dispense the agent for delivery to the individual, and a controller coupled to the reservoir and the dispenser and configured to execute instructions for delivery of the agent to the individual at a predefined time and a predefined dosage. The computing device may include a memory configured to store instructions and a processor coupled to the memory. The processor in conjunction with the instructions may be configured to provide the instructions for delivery of the agent to the individual at the predefined time and dosage to the controller. Following the delivery of the agent to the individual, the processor may be configured to receive an input associated with one or more parameters related to a physiological function and/or a cognitive function of the individual. The processor may then be configured to process the input to analyze an efficacy of the agent with respect to the physiological and/or cognitive functions of the individual, and adjust the predefined time and/or dosage for delivery of the agent to the individual based on the analysis.

According to yet further examples, delivery devices configured to deliver agents are described. An example delivery device may include a reservoir configured to hold an agent, a dispenser configured to receive the agent upon release from the reservoir and dispense the agent for delivery to an individual, and a controller coupled to the reservoir and the dispenser and configured to execute instructions received from a processor to deliver the agent to the individual at a predefined time and a predefined dosage. The predefined time and dosage may be adjusted based on an analysis of an efficacy of the agent with respect to a physiological function and/or a cognitive function of the individual performed by the processor.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 includes a conceptual illustration of an example method to control and optimize a timing and dosage of agents delivered to an individual through a delivery device;

FIG. 2 includes an example delivery device integrated in a wearable device;

FIG. 3 includes an example self-adhering delivery device;

FIG. 4 includes an example delivery device integrated in a clothing item;

FIG. 5 includes a conceptual illustration of an example apparatus configured to control and optimize agent delivery to an individual in a tailored manner;

FIG. 6 includes a conceptual illustration of timing and dosage adjustment of an agent based on analysis of input retrieved prior to and following the delivery of the agent;

FIG. 7 includes a conceptual illustration of schedule variability for a given agent;

FIG. 8 includes a conceptual illustration of timing and dosage adjustment of multiple agents based on an efficacy analysis of the agents, individually and in combination;

FIG. 9 illustrates major components of an example system configured to control and optimize delivery of agents to the individual in a tailored manner;

FIG. 10 illustrates a computing device, which may be used in conjunction with a delivery device to control and optimize delivery of an agent to an individual in a tailored manner;

FIG. 11 is a flow diagram illustrating an example method to control and optimize delivery of an agent to an individual in a tailored manner performed by a computing device such as the computing device in FIG. 7 in conjunction with a delivery device such as the delivery device in FIG. 1; and

FIG. 12 illustrates a block diagram of an example computer program product, some of which are arranged in accordance with at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. The aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems, devices, and/or computer program products related to controlling delivery of agents to an individual.

Briefly stated, technologies are generally described for controlling and optimizing delivery of agents to an individual through a delivery device in a tailored manner. The delivery device may include reservoirs that hold the agents and dispensers that, upon release from the reservoirs, receive and dispense the agents to the individual, where a controller coupled to the reservoirs and dispensers may be configured to control the delivery of the agents at a predefined timing and dosage. Following delivery of the agents, input, such as sensor and/or data feed associated with parameters related to the individual's physiological and/or cognitive functions, may be received from sensors, computing devices, smart devices, and networks of the individual. The input may be processed to analyze an efficacy of the agents with respect to the individual's physiological and/or cognitive functions. The analysis may include a comparison of the processed input to a baseline profile of the individual defined prior to the delivery of the agents to determine whether the individual's physiological and/or cognitive functions have improved, declined, or remained the same. The input may be processed to analyze efficacy of each agent individually, as well as to determine interactions among the agents, in combination. The predefined timing and dosage may then be adjusted for a next delivery of the agents based on the analysis of the efficacy of the agents and any determined interactions.

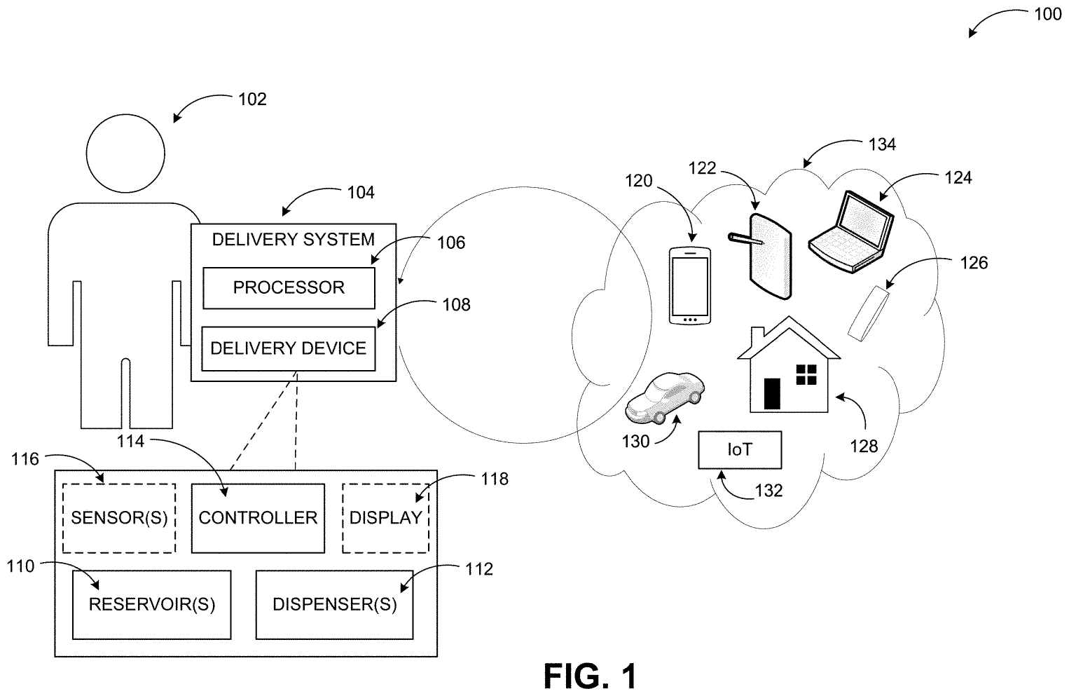

FIG. 1 includes a conceptual illustration of an example method to control and optimize a timing and dosage of agents delivered to an individual through a delivery device.

In an example scenario, an individual may be on a nootropics regimen. Such a regimen may be intended to help healthy individuals improve their cognitive performance, rather than act as a treatment for a deficiency or disorder, for example. This typically involves delivery of multiple different agents at varying times and dosages, which is often referred to as a full stack. These agents may include compounds, extracts or mixtures, among other examples that effect various aspects of cognitive function such as executive functions (i.e., attentional control, inhibitory control, working memory, cognitive flexibility, reasoning, problem solving and planning), memory, creativity and/or motivation. Currently, most of these agents are either in pill or powder form that are taken orally, and thus a full stack program typically requires a person to take multiple pills at different times of the day. This may be very difficult for the individual to self-manage. Pills may be forgotten or taken at the incorrect times, which may render the agents individually or in combination less effective. Additionally, too many pills or pills in an incorrect combination may be taken, which may potentially lead to harmful effects. Embodiments as described herein, relieve such difficulty and potential for error leading to inefficacy or harmful effects by automatically controlling the delivery of the multiple agents to the individual through a delivery device. Moreover, the embodiments provide individually-tailored optimization of the timing and dosage for the delivery of the agents based on sensor and data feed related to physiological and/or cognitive functions of the individual in order to increase an efficacy of each agent, individually and in combination with the other agents, with respect to that individual.

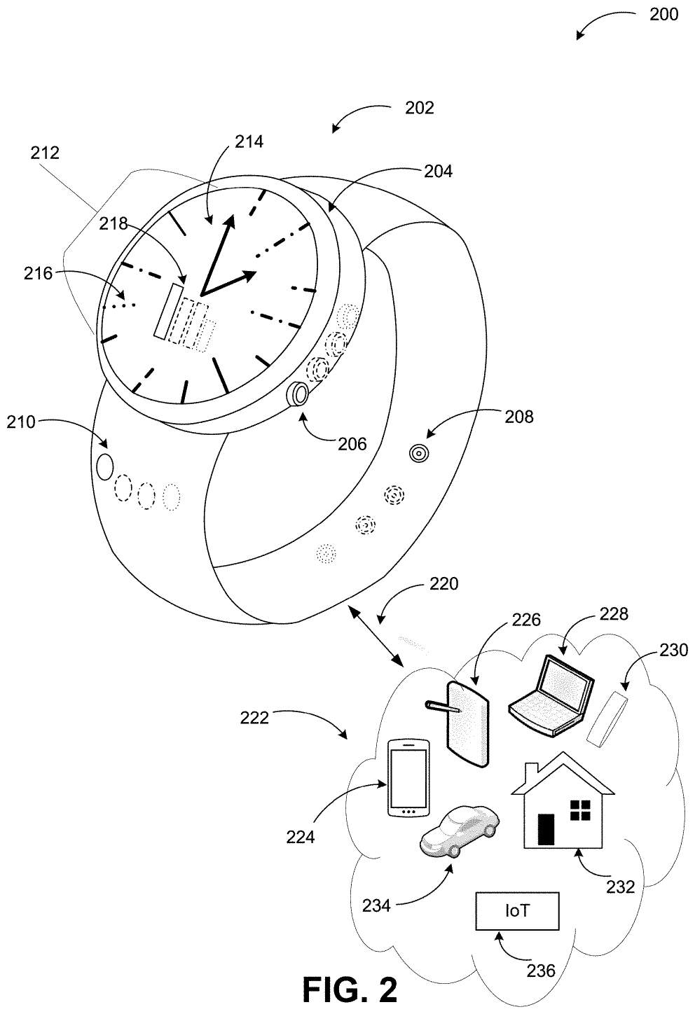

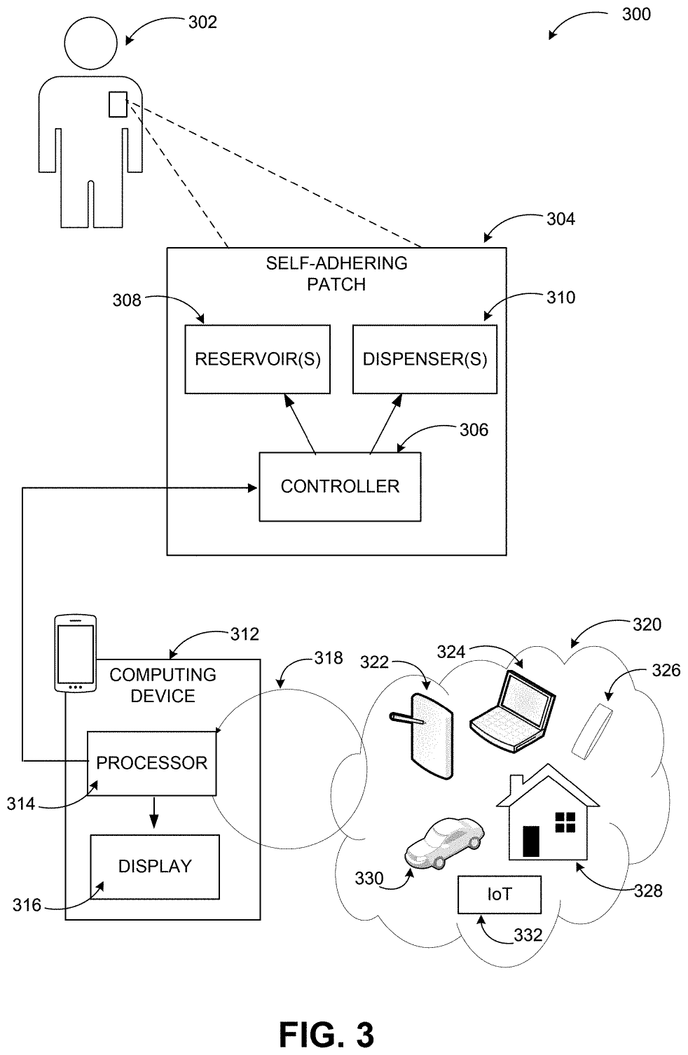

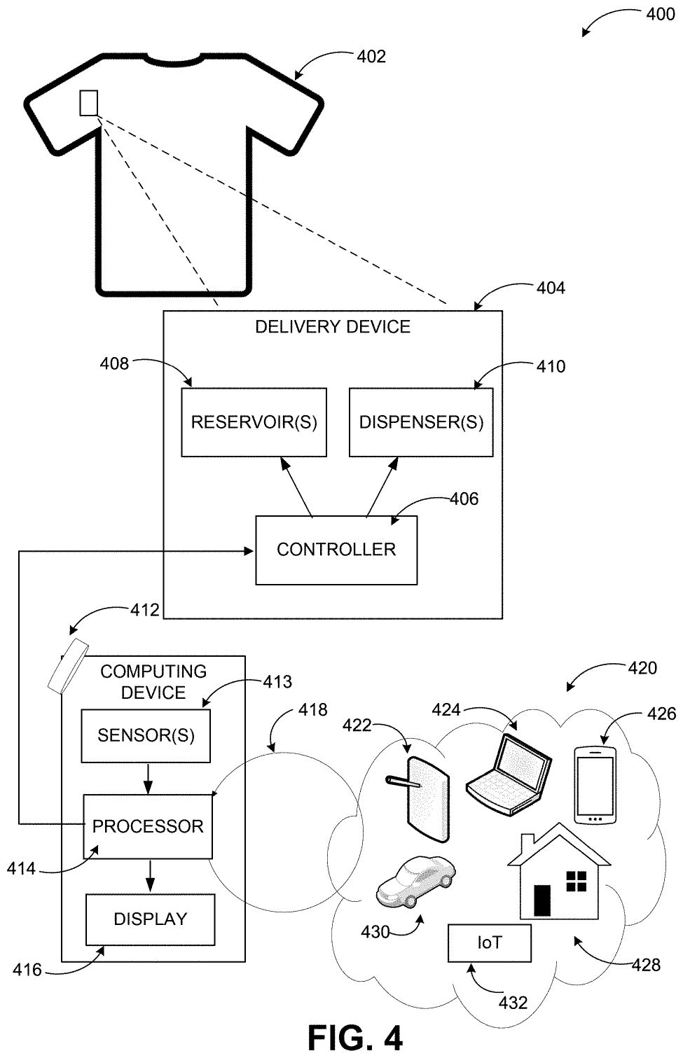

For example, as shown in a diagram 100, a delivery system 104 may include at least a processor 106 and a delivery device 108 configured to deliver one or more agents to an individual 102. The agents may include drugs, supplements, or cognitive-enhancing substances, such as a nootropic, as described in the example scenario above. The delivery device 108 may be a transdermal delivery device implemented in variety of forms, including but not limited to, a device strapped, adhered, or otherwise affixed or attached to the body, such as a smart watch as illustrated in FIG. 2 and a self-adhering device as illustrated in FIG. 3, or a device integrated into clothing items as illustrated in FIG. 4. Additionally, the delivery device 108 may be integrated in an everyday item the individual 102 tactilely interacts with. For example, the delivery device 108 may be implemented as a wall mount in the individual's home or office or may be integrated into commonly touched objects such as a coffee mug, a remote, a computer mouse, a cell phone case, and furniture, among other similar objects. For transdermal delivery, the agents may either be naturally available when applied through the skin of the individual 102, or formulated in such a way as to be able to penetrate the skin (e.g., through encapsulation strategies such as liposomes). In other embodiments, the delivery device 108 may be an oral delivery device, a nasal delivery device, a sublingual delivery device, a buccal delivery device, an ocular delivery device, an otic delivery device, a rectal delivery device, a vaginal delivery device, an intravenous delivery device, an intramuscular delivery device, a subcutaneous delivery device, an intradermal delivery device, an intrathecal delivery device, a cutaneous delivery device, a nebulizer, or an inhaler.

The delivery device 108 may include at least one or more reservoirs 110, one or more dispensers 112, and a controller 114. The reservoirs 110 may be configured to hold the agents and the dispensers 112 may be configured to receive the agents upon release from the reservoirs 110 and dispense the agents to the individual 102. The controller 114 may be coupled to the reservoirs 110 and the dispensers 112 and may be configured to control the delivery of the agents to the individual 102 at a predefined timing and dosage. The predefined timing and dosage may indicate a time of day for delivery, a frequency of delivery, a rate of delivery, a number of dosages to be delivered, and/or an actual amount or size of the agent delivered, among other examples. For example, the controller 114 may receive instructions regarding the delivery of the agents at the predefined timing and dosage from the processor 106. The processor 106 may be a component of a separate computing device or server. Alternatively, the processor 106 may be a component of the delivery device 108.

The reservoirs 110 may be refillable and, in some embodiments, one or more of the reservoirs 110 may include replaceable cartridges configured to hold the agent. Depending on the regimen to be provided to the individual 102, each of the reservoirs 110 or cartridges may hold a different type of agent and/or a different dosage of agent. Each of the reservoirs 110 or the cartridges may also include a level sensor configured to provide an alert when an amount of the agent remaining in the respective reservoir or cartridge is below a predetermined threshold.

The dispensers 112 may be microdispensers that receive the agents after the controller 114 prompts the release of the agents from the reservoirs 110 or cartridges. Depending on the regimen to be provided to the individual 102, each of the dispensers 112 may dispense a different type of agent and/or a different dosage of agent, similar to the reservoirs 110 and cartridges. For example, each one of the dispensers 112 may correspond to a particular one of the reservoirs 110 or cartridges to prevent mixing and/or cross-contamination of agents in subsequent deliveries of the agents. The dispensers 112 may include embedded sensors that monitor a status of the dispensers 112 and provide an alert if any malfunction is detected.

In some embodiments, the delivery device 108 may be a transdermal delivery device and the dispensers 112 may dispense the agent onto skin of the individual 102 or onto a substrate, such as a wicking pad, positioned on the skin of the individual 102 to enable transdermal delivery of the agent. In other embodiments, the agent may be delivered through other routes, including, but not limited to, oral, nasal, sublingual, buccal, ocular, otic, rectal, vaginal, intravenous, intramuscular, subcutaneous, intradermal, intrathecal, cutaneous, nebulization, or inhalation routes, based on a type of the delivery device.

In some embodiments, the dispensers 112 may be microdispensers configured to dispense agents in a liquid state when energy, in the form of heat, is applied to a reservoir containing the liquid agent thereby causing the liquid agent in contact with sides of the reservoir to form a vapor bubble. The formation and collapsing cycle of a vapor bubble may create a one-directional dispensing action. Dependent on a form in which the delivery device is implemented, the reservoir may be one of the above-discussed reservoirs 110 or the reservoir may be a separate reservoir component of the microdispensers, for example. The microdispensers may be heated by resistive (i.e. joule) heating. For example, a voltage may be applied to a resistor for a few micro-seconds. Subsequently, the resistor rapidly heats up to a high temperature, and nucleates a bubble at the fluid contact points. The bubble grows explosively, thereby causing the inertial dispensing and then collapses. Assuming 50 ohms (Ω) per resistor and a 10-volt (V) supply to create a mature bubble, the power consumption may be approximately 2 watts (W). A single resistor may create a bubble the size of 50 picoliters (pL). However, the multiple resistors can be combined. For example, one hundred resistors firing simultaneously may create 5,000 pL for each sequence. Assuming they are firing at 5 kilohertz (KHz) or 5,000 events per second, then the flow rate of the liquid agent will be 25 μL per second as it is dispensed from the microdispensers to the individual 102.

Following the delivery of the agents to the individual 102, the processor 106 may receive input, such as sensor and data feed, associated with parameters related to the individual's physiological and/or cognitive functions. The input may include the individual's temperature, heart rate, heart electrical activity, muscle electrical activity, sleep and wake patterns, diet, movement patterns, reaction times, responsiveness, speed and error rates associated with tasks, and results of cognitive assessments, among other examples. In some embodiments, the delivery device 108 may include one or more sensors 116 from which input is received. For example, the sensors 116 may include accelerometers to measure movement during wake and sleep cycles, audio sensors such as microphones, visual sensors such as cameras or light sensors, gyroscopes, and biometric sensors such as a thermometer, a heart rate monitor, an electrocardiogram, or electromyographic sensors. Additionally, the input may be received from computing devices, smart devices, and/or networks associated with the individual, such as a cell phone 120, a tablet 122, a laptop 124, a wearable device 126, smart home devices 128, smart transportation devices 130, and an Internet of things (IoT) network 132, among other examples. These computing devices, smart devices, and/or networks associated with the individual may be communicatively connected wired or wirelessly to the delivery system 104 over one or more networks, such as cloud network 134. The input received from these devices and/or networks may include sensor-based data, similar to the data provided by the sensors 116, in addition to application or network based data that may be associated with parameters related to or indicate events that may affect the physiological and/or cognitive functions of the individual 102. For example, calendar related data may be received from a calendar and/or communication application executing on the cell phone 120, the tablet 122 or the laptop 124. These calendar records may include planned sleep times, meetings or exam times where alertness or cognitive functioning is particularly important and stress levels may be elevated, exercise programs, or travel, among other similar activities that may require a proactive adjustment in a timing and a dosage of the agent. Similar data may be retrieved from professional or social networking applications based on events the individual indicated he or she would attend, and comments or posts regarding upcoming tasks or travel, among other examples.

In an example scenario, the wearable device 126, may measure and provide data related the overall health and sleep patterns of the individual 102. The smart home devices 128 may measure and provide data related to the person's diet, movement patterns, sleep and wake patterns, general health, and activity of the individual 102. The smart transportation devices 130 may provide measurements and data related to reaction times and responsiveness of the individual 102. The cell phone 120, the tablet 122, and the laptop 124 may measure and provide data related to error rates for spelling, attention span, focus, and task achievements, among other examples. The above-provided examples, illustrate passive gathering of the input. Alternatively, data received as input may be in response to actively directed tests that are designed to measure a specific cognitive function of the individual 102. For example, the tests may be provided to the individual 102 to perform through the delivery device 108 itself or the tests may be presented to the individual 102 through an application being executed on one of the computing devices associated with the individual 102, such as the cell phone 120, the tablet 122, or the laptop 124.

In some embodiments, the delivery system 104 may provide at least a portion of the received input to one or more other medical or health related devices within a connected health system, for example. In further embodiments, the delivery system 104 may interact with drones to obtain additional input or data (e.g., environmentally-related data discussed below) and/or to re-supply the delivery device 108 with the agents, among other examples.

The processor 106 may process the input to analyze an efficacy of each of the agents with respect to the individual's physiological and/or cognitive functions. The input may be processed in several ways so that both short-term and long-term effects of the agents may be accounted for when optimizing the timing and dosage of the agents for delivery to the individual 102. In some embodiments, the processor 106 may employ a least squares approach when processing the input. A least squares approach may determine a best fit line to data using simple calculus and linear algebra. Due to the simplicity of the least squares approach, availability of accurate data representing the physiological and/or cognitive functions being targeted is critical. Therefore, it may be optimal to have a plurality of devices, sensors, and data feeds providing input for processing.

The efficacy analysis may include a comparison of the processed input to a baseline profile of the individual's physiological and/or cognitive functions defined prior to the delivery of the agents to determine whether the functions have improved, declined, or remained the same following the delivery of the agents. In addition to processing the input to analyze efficacy of each agent individually, the input may be further processed to determine interactions among the agents, such as by performing analysis of variance (ANOVA) analysis or using artificial intelligence methods. ANOVA is a collection of statistical models and their associated procedures used to analyze the differences among group means. ANOVA may be particularly useful for comparing three or more means (groups or variables) for statistical significance.

The processor 106 may then adjust the predefined timing and/or dosage at which one or more of the agents were delivered for a next delivery based on the efficacy analysis and any determined interactions among the agents. Adjustments may be made to the time of day of delivery, the frequency of delivery, the rate of delivery, the number of dosages delivered, and/or the actual amount or size of the agent delivered, among other similar adjustments. For example, if the functions associated with a particular agent have declined, the predefined timing and/or dosage may be decreased for the agent. If the functions have improved, the predefined timing and/or dosage may be increased or maintained depending on whether the predefined timing and/or dosage is already at an upper limit threshold for the type of the agent. If the functions have remained the same, additional time may be given for the agent to take effect. After a particular waiting period, another input will be received from the sensors 116 and computing devices, smart devices, and/or networks associated with the individual. The other input received after the waiting period may include similar sensor and data feed to the input received following the delivery of the agents. The other input may be processed and compared to the baseline profile to determine whether the functions have improved, declined, or remained the same following the delivery of the agents and after the particular waiting period.

In some embodiments, data related to an environment of the individual may also be received. The processor 106 may determine whether the environment-related data indicates the physiological and/or cognitive functions of the individual may be affected. Upon a positive determination, the processor 106 may further adjust the predefined time and/or dosage at which the agent is to be delivered to the individual based on the environment-related data. For example, if the environment-related data yields an increased pollen count emerging, and one of the agents includes an allergy drug, then a dosage of the allergy drug may be proactively increased before the onset of the biological reaction to the individual 102. In other embodiments, data related to the efficacy of the agent with respect to a physiological function and/or a cognitive function of a plurality of individuals may be received. The predefined time and/dosage at which the agent is to be delivered to the individual may then be adjusted based on both the analysis and the data. For example, population level data from the plurality of individuals may further inform optimized time and dose scheduling from a larger data pool.

The above-described process of agent delivery, input processing and analysis, and time and/or dosage adjustment based on the analysis may be continuously repeated. Through this iterative process, the delivery system 104 may learn the most effective timing and dosage for each agent individually, and in combination with the one or more other agents, thus optimizing the outcome of the regimen.

In further embodiments, the delivery device 108 may include a display 118. The display 118 may present information associated with a timing and a dosage at which the agent is delivered to the individual (e.g., the predefined timing and dosage or a later adjusted timing and dosage). The display 118 may also present performance results based on the analysis of the efficacy of the agent. In some examples, if the input indicates the individual 102 should seek medical attention, the delivery system 104 may prompt the individual to do so through the display 118 of the delivery device 108. For example, an alert and/or notification may be displayed.

FIG. 2 includes an example delivery device integrated in a wearable device. As shown in a diagram 200, a delivery device may be a transdermal delivery device that is integrated in a wearable device, such as a smart watch 202. The smart watch 202 may be configured to control and optimize delivery of one or more agents to an individual in a tailored manner.

A case 204 of the smart watch 202 may contain, among other things, a processor, a controller, and a power supply of the smart watch 202. The power supply may be a rechargeable battery that may be remotely charged on an induction charger, for example. Alternatively, the power supply may include a motion capture device, or a solar power converter, among other examples. In some embodiments, the processor may prompt the controller to automatically activate the power supply in response to detecting a proximate position of the smart watch 202 to the individual. For example, upon detecting contact between the smart watch 202 and skin of the individual. Similarly, the processor may prompt the controller to automatically deactivate the power supply in response to detecting a removal of the smart watch 202 from the proximate position to the individual.

A band 207 attached to the case 204 may affix the smart watch 202 to the individual's wrist, for example. The controller may be coupled to reservoirs 206 located in an exterior portion of the case 204 and dispensers 208 located in an interior portion of the band 207. The processor may determine a timing and a dosage for delivery of each of the agents. The predefined timing and dosage may indicate a time of day for delivery, a frequency of delivery, a rate of delivery, a number of dosages to be delivered, and/or an actual amount or size of the agent delivered, among other examples. The processor may then provide instructions to the controller for delivery of the agents at a respective predefined timing and dosage. For example, the controller may prompt a release of the agents from the reservoirs 206 to the dispensers 208, and a subsequent dispense of the agents from the dispensers 208 to the individual's skin at the respective predefined timing and dosage. Each of the dispensers 208 may correspond to a particular one of the reservoirs 206 to prevent mixing and/or cross-contamination of the agents.

In some embodiments, sensors 210 may span a thickness of the band 207 of the smart watch 202 such that portions of the sensors 210 on an interior surface of the band 207 may be in contact with the individual's skin and portions of the sensors 210 on an exterior surface of the band 207 may be in contact with the individual's environment. The sensors 210 may include accelerometers, audio sensors, visual sensors, gyroscopes, and/or biometric sensors, among other examples.

Following the delivery of the agent, the sensors 210 may provide data to the processor related to the individual's physiological and/or cognitive functions and data related to the individual's environment. Additionally, the processor may receive input 220 from computing devices, smart devices, and networks associated with the individual, such as a cell phone 224, a tablet 226, a laptop 228, and a wearable device 230, smart home devices 232, smart transportation devices 234, and an Internet of things (IoT) network 236. These computing devices, smart devices, and networks associated with the individual may be communicatively connected wired or wirelessly to the smart watch 202 over one or more networks, such as cloud network 222. The input 220 may include sensor-based data, similar to the data provided by the sensors 210, in addition to application or network based data that may be associated with parameters related to or indicate events that may affect the physiological and/or cognitive functions of the individual.

In an example scenario, the sensors 210 on the smart watch 202 may measure and provide data related to the individual's heart rate as well as the general movement, exercise, and sleep patterns of the individual. A smart home device, such as a smart television located in the individual's home, may measure and provide data related to the individual's activity in association with the smart television. For example, the measurements may include an amount of time the individual is watching the television, and the individual's focus and/or attention span during that time based on eye gaze data. A smart transportation device located in the individual's car may provide measurements and data related to reaction times and responsiveness of the individual while driving. For example, the measurements may include an amount of time it takes the individual to stop the car upon visualizing a red stop light. The laptop 228 may be executing a word-processing application, and may measure and provide data related to error rates for spelling and attention span based on eye gaze and frequency of interaction with the application (e.g., frequency of typing, selecting of other inputs, etc.). The smart watch 202 itself may also prompt the individual to perform a memory test through a display 212 of the smart watch 202 to assess how the individual's current working memory is functioning. In addition to providing data associated with parameters directly related to the individual's physiological and cognitive functions, data related to events that may indicate a potential effect on the individual's physiological and cognitive functions may also be provided. For example, the individual may post a status through a social network (i.e., within the IoT network 236) complaining about the four exams he or she has next week. Exams are events that may induce stress and disrupt sleep and other activity patterns due to the studying required, which likely effects the individual's physiological and cognitive functions. Thus, knowledge of this event may enable proactive adjustment of timing and/or dosage of one or more of the agents to combat those effects.

The processor may process the data from the sensors 210 along with the input 220 to analyze an efficacy of the agent with respect to the individual's physiological and cognitive functions. For example, the analysis may include a comparison of the processed input to a baseline profile of the individual's physiological and/or cognitive functions defined prior to the delivery of the agents to determine whether the functions have improved, declined, or remained the same following the delivery of the agents. The baseline profile may be defined using similar data from the sensors 210 and input 220 received prior to the delivery of the agents. In addition to processing the input to analyze efficacy of each agent individually, the input may be further processed to determine interactions among the agents. The predefined timing and/or dosage at which the agent was delivered may then be adjusted for a next delivery based on the analysis and any determined interactions to optimize efficacy of the agents, individually and in combination, as to the individual. In some embodiments, the processor may be further configured to determine whether the environment-related data obtained from sensors 210, for example, indicates the physiological and/or cognitive functions of the individual may be affected. Upon a positive determination, the processor may further adjust the predefined time and/or dosage at which the agent is to be delivered to the individual based on the environment-related data.

As previously discussed, the smart watch 202 may also feature a display 212 on a face of the smart watch 202. The display 212 may include an analog clock 214 and visual representations 216 of a type and dosage of an agent in conjunction with a time on the analog clock 214 that the agent was or will be delivered. For example, in the visual representations 216, each type of agent delivered may be associated with a particular color, a size of bar indicators in the particular color may represent a dosage of the respective type of agent delivered, and the timing of delivery may be represented by placing the bar indicators at a position on the analog clock 214 to illustrate when the agent is delivered at that particular dosage. Additionally, the display may present performance results 218 based on the efficacy analysis. Efficacy analysis may be performed through cognitive functionality testing in some cases. Following are non-limiting examples of cognitive tests that may be used in conjunction with a device, system, or method according to embodiments.

### Inductive Reasoning Testing

One approach to measure the efficacy of a nootropics program may be testing a person's inductive reasoning by measuring how well they can identify a pattern with a large amount of data. This may be done by presenting the test taker with a series of four pictures or words and asking them to identify which does not belong in the set. A typical test may be conducted by presenting several of these sets of different degrees of difficulty and measuring how many of these a person can properly identify in a set period of time. An example set may be:

Find the group of letters that does not belong with the other groups:
(a) cdfg
(b) lmnp
(c) ghkj
(d) uwzx

* Correct answer is . . . (d) as it is the only group containing a vowel

Another, slightly more difficult, example may be:

Find the group of letters that doesn't belong with the other groups:
(a) cdef
(b) mnpo
(c) hikj
(d) uwvx

* Correct answer is . . . (a) as it is the only group with four letters in sequential order

In some examples, the person may be wearing a smartwatch or have a smartphone to present the test and measure the outcome. They may periodically get asked by the system to complete the inductive reasoning test, to measure the efficacy of the nootropics program. This may involve the person electing to start the test, which may give the person a predefined (e.g., two) minutes to complete as many of these inductive reasoning tasks as possible. The sets may be presented on the smartphone or smartwatch one at a time during the test, and the person answers them, to proceed to the next set. The sets may be randomly assigned from a larger data set, and/or may escalate in difficulty. The system may then determine whether the person's current score (that is, a number of correct answers completed in the time period) is higher or lower than their previous score or a baseline they established at the beginning of their nootropics program. Multiple tests over time may add to the robustness of this measurement and enable statistical analysis to determine trends over time with the program, such as by measuring the variance of the number of correct answers in two-minute tests over time, such as with an ANOVA analysis. This may also be useful for modifying the nootropics program over time to optimize effective dose scheduling for an individual.

### Short Term Memory Testing

One method to assess short term memory is a digit span test. This may be provided visually or auditorily (for example, presented to the person via smartwatch, smartphone, or on a computer) as a sequence of numerical digits with increasingly longer sequences being tested in each trial. The person may then be tasked to recall the sequence, in forward and/or reverse order. The person's span is the longest number of sequential digits that can accurately be remembered. For the purpose of monitoring and/or modulating the efficacy of a nootropics program, as described herein, such a test may be useful to track short term memory changes over time. For example, the person may be subjected to these tests, initially to establish a baseline of their mean digit span recall (that is, the average of the longest number of digits able to be recalled from a sequence) and typical variance thereof. Then, the test may be repeated periodically thereafter to determine if changes have occurred to either the mean. Repeated measures (e.g., daily or weekly) may be useful to provide statistical power to this assessment, and allow for determination if changes are statistically significant, such as by providing an average and standard deviation used in t-tests or ANOVA analysis. In addition to measuring working memory's number storage capacity, performance on the digit-span task is a measure of verbal working memory, and is also closely linked to language learning abilities.

### Wechsler Adult Intelligence Scale (WAIS)

The WAIS is a standardized intelligence quotient (IQ) test designed to measure intelligence and cognitive ability in adults and older adolescents. The latest version, WAIS-IV was released in 2008 and is composed of 10 core subtests and five supplemental subtests providing four index scores representing major components of intelligence: verbal comprehension, perceptual reasoning, working memory, processing speed. Two broad scores may be used from this to summarize general intellectual abilities, Full Scale IQ (FSIQ) and General Ability Index (GM). For the purpose of monitoring the efficacy of a nootropics program, the person may be presented with the WAIS to establish a baseline FSIQ and/or GM, and then periodically retested to determine if there has been any change to these scores. The retesting may be done weekly or monthly, and may be done as the whole WAIS, or just partial testing which may enable more frequent testing. The tests may be administered via the person's smartphone or computer at scheduled times or at will.

### Attention Monitoring

Attention is the behavioral and cognitive process of selectively concentrating on a discrete aspect of information, while ignoring other perceivable information. Driving a car requires significant attention to ensure safety, and driver attention has been the subject of numerous studies. It is possible to monitor the attentive state of a driver using computer vision techniques by both standard and day-night infrared cameras. Signs such as visual distraction, like off-road gaze direction and persistent rotation of the head, as well as, changes in facial features which characterize persons with reduced alertness due to fatigue: longer blink duration, slow eyelid movement, small degree of eye opening, nodding, yawning and drooping posture are among the those that may be useful to monitor attention. The attention monitoring may be performed in a relatively autonomous fashion with a camera-equipped car, for example, using common processing schemes that include the following steps: face localization, localization of facial features (eyes, mouth, etc.) estimation of specific cues related to fatigue or distraction, fusion of cues in order to determine the global attention level. The monitoring may be performed for a period of time, such as a week or a month, for the person in order to establish a baseline before starting a nootropics program. Then, the global attention level may be measured every time they drive to determine if changes are occurring. If necessary, the nootropics program can be modified based on the monitoring.

FIG. 3 includes an example self-adhering delivery device. As shown in a diagram 300, a delivery device may be a transdermal delivery device that is self-adhering to skin of an individual 302, such as a patch 304. For example, the patch 304 may be in a blister pack style that includes one or more reservoirs 308, one or more dispensers 310, and a controller 306. The controller may be coupled to the reservoirs 308 and dispensers 310. The patch 304 may adhere to the individual's skin at various sites, such as on the wrist, arm, shoulder, chest, back, leg, abdomen, and neck, among other examples. The patch 304 may be configured to deliver one or more agents to the individual 302 in conjunction with a computing device 312 associated with the individual 302, such as a cell phone. In this example scenario, a single type of agent may be delivered. In other examples, multiple different types of agents may be delivered.

The computing device 312, may be communicatively coupled to the controller 306 of the patch 304 using near field protocols or over a network, such as a wireless local area network or cellular network. The computing device may include, among other things a processor 314. The processor 314 may define a timing and a dosage for delivery of an agent to the individual 302, The predefined timing and dosage may indicate a time of day for delivery, a frequency of delivery, a rate of delivery, a number of dosages to be delivered, and/or an actual amount or size of the agent delivered, among other examples. The processor 314 may then instruct the controller 306 to deliver the agent at the predefined timing and dosage. For example, the controller 306 may prompt a release of the agent from the reservoirs 308 to the dispensers 310, and a subsequent dispense of the agent from the dispensers 310 to the individual 302 at the predefined timing and dosage. The agent may be dispensed directly onto the individual's skin or onto a wicking pad, for example, positioned between the dispenser 310 and the individual's skin.

Following delivery of the agent, the processor 314 may receive input 318 such as sensor and data feed that may be associated with parameters related to or indicate events that may affect the physiological and/or cognitive functions of the individual 302. The input 318 may be received from computing devices, smart devices, and/or networks associated with the individual, such as a tablet 322, a laptop 324, a wearable device 326, smart home devices 328, smart transportation devices 330, and an Internet of things (IoT) network 332. These computing devices, smart devices, and/or networks associated with the individual may be communicatively connected wired or wirelessly to the computing device 312 over one or more networks, such as cloud network 320. The input may also include sensor and data feed obtained from the computing device 312 itself.

In an example scenario, the computing device 312 may measure and provide data related to attention span, focus, and task achievements, among other examples. For example, the computing device 312 may use its cameras and/or light sensors to track an eye gaze of the individual to determine attention span and focus. Additionally, the computing device 312 may monitor a completion of tasks of the individual through a communication application, for example, executed on the computing device 312. The wearable device 326 may be a fitness device that measures and provides data related to activity level, caloric input and output, and heart rate, for example. A smart home device, such as a smart security system comprising motion sensors within the individual's home, may measure and provide data related to the individual's movement patterns, sleep and wake patterns, and activity of the individual 302 within the home. A smart transportation device located in the individual's car may provide measurements and data related to an attention span of the individual while driving. For example, the measurements may include how frequently and for what length of time the individual 302 is not looking ahead at the road and surrounding traffic, but is instead looking at other passengers, devices, and/or components of the car such as the radio or car environment controls, among other examples. In addition to providing data associated with parameters directly related to the individual's physiological and cognitive functions, data related to events that may indicate a potential effect on the individual's physiological and cognitive functions may also be provided. For example, information may be extracted from a calendar application that is being executed on one or more of the individual's devices, such as the tablet 322 or the laptop 324. The calendar may indicate the individual 302 is scheduled to lead a meeting early tomorrow morning. Because the individual 302 is leading the meeting, and at such an early hour, it may be beneficial for the individual 302 to be more alert than normal. Thus, knowledge of this event may enable proactive adjustment of timing and/or dosage of one or more of the agents to enable increased alertness.

The processor 314 may process input 318 to analyze an efficacy of the agent with respect to the individual's physiological and cognitive functions. For example, the analysis may include a comparison of the processed input 318 to a baseline profile of the individual's physiological and/or cognitive functions defined prior to the delivery of the agents to determine whether the functions have improved, declined, or remained the same following the delivery of the agents. The predefined timing and/or dosage at which the agent was delivered may then be adjusted for a next delivery based on the analysis to optimize efficacy of the agent as to the individual.

The computing device may also include a display 316. The display may include information about a type and a dosage of the agent to be delivered in conjunction with the time that the agent was or will be delivered. Additionally, the display 316 may present performance results based on the efficacy analysis.

FIG. 4 includes an example delivery device integrated in a clothing item. As shown in a diagram 400, a delivery device 404 may be integrated in a clothing item 402, such as shirt, worn by an individual. Other clothing items in which the delivery device 404 may be integrated include hats, belts, scarves, pants, and shoes, among other examples.

The delivery device 404 may include reservoirs 408, dispensers 410, and a controller 406 coupled to the reservoirs 408 and dispensers 410. The delivery device 404 may be configured to control delivery of one or more agents to the individual in conjunction with a computing device 412 of the individual, such as a wearable device. In this example scenario, a single type of agent may be delivered. In other examples, multiple different types of agents may be delivered.

The computing device 412 may be communicatively coupled to the delivery device 404 using near field protocols or over a network, such as a wireless local area network or cellular network. The computing device 412 may include, among other things a processor 414. The processor 414 may define a timing and a dosage for delivery of an agent to the individual that indicates a time of day for delivery, a frequency of delivery, a rate of delivery, a number of dosages to be delivered, and/or an actual amount or size of the agent delivered, among other examples. The processor 414 may then instruct the controller 406 to deliver the agent at the predefined timing and dosage. For example, the controller 406 may prompt a release of the agent from the reservoirs 408 to the dispensers 410, and a subsequent dispense of the agent from the dispensers 410 to the individual at the predefined timing and dosage. The agent may be dispensed directly onto the individual's skin or onto a wicking pad, for example, adhered to a portion of the individual's skin in alignment with the delivery device 404.

The computing device 412 may also include one or more sensors 413. The sensors 413 may include accelerometers, audio sensors, visual sensors, gyroscopes, and/or biometric sensors, among other examples. Following the delivery of the agent, the sensors 413 may provide data to the processor related to the individual's physiological and/or cognitive functions. Additionally, the processor may receive input 418 may from other computing devices, smart devices, and networks associated with the individual, such as a tablet 422, a laptop 424, a cell phone 426, smart home devices 428, smart transportation devices 430, and an Internet of things (IoT) network 432. These other computing devices, smart devices, and networks associated with the individual may be communicatively connected wired or wirelessly to the computing device 412 over one or more networks, such as cloud network 420. The input 418 may include sensor-based data, similar to the data provided by the sensors 413, in addition to application or network based data that may be associated with parameters related to or indicate events that may affect the physiological and/or cognitive functions of the individual.

In an example scenario, the computing device 412 may measure and provide data related to the individual's overall health and fitness, such as activity level, heart rate, and caloric input and output, as well as sleep patterns of the individual. A smart home device, such as a smart refrigerator, may measure and provide data related to the individual's dietary choices within the home. For example, the data may include a type and/or amount of food consumed by the individual. A smart transportation device located in the individual's car may provide measurements and data related to alertness of the individual while driving. For example, the individual's eye gaze, posture, and positioning of hands on the wheel, among other examples, may be monitored and measured by one or more sensors within the car to determine how awake and alert the individual is. The cell phone 426 may be prompted by the computing device to provide the individual a cognitive assessment through a gaming application, for example, and the cell phone 426 may provide data related to the results of the cognitive assessment. In addition to providing data associated with parameters directly related to the individual's physiological and cognitive functions, data related to events that may indicate a potential effect on the individual's physiological and cognitive functions may also be provided. For example, information may be extracted from an invite accepted through a social network (i.e., within the IoT network 432) to a late-night party hosted by friends of the individual. The party may be occurring during hours in which the individual is usually not active or is sleeping. Thus, knowledge of this event may enable proactive adjustment of timing and/or dosage of one or more of the agents to increase energy and alertness in order to stave off normal inactivity and sleep patterns of the individual.

The processor 414 may process the data from the sensors 413 and the input 418 to analyze an efficacy of the agent with respect to the individual's physiological and cognitive functions. For example, the analysis may include a comparison of the processed input 418 to a baseline profile of the individual's physiological and/or cognitive functions defined prior to the delivery of the agents to determine whether the functions have improved, declined, or remained the same following the delivery of the agents. The predefined timing and/or dosage at which the agent was delivered may then be adjusted for a next delivery based on the analysis to optimize efficacy of the agent as to the individual.

The computing device may also include a display 416. The display 416 may include information about a type and a dosage of the agent to be delivered in conjunction with the time that the agent was or will be delivered. Additionally, the display 416 may present performance results based on the efficacy analysis.

The various hardware implementations of the delivery device include but are not limited to the example transdermal delivery devices, described in conjunction with FIGS. 2 through 4. Additional implementations of transdermal delivery devices may include integration of the delivery device in an everyday item the individual tactilely interacts with. Alternatively, in other embodiments, the delivery device may be an oral delivery device, a nasal delivery device, a sublingual delivery device, a buccal delivery device, an ocular delivery device, an otic delivery device, a rectal delivery device, a vaginal delivery device, an intravenous delivery device, an intramuscular delivery device, a subcutaneous delivery device, an intradermal delivery device, an intrathecal delivery device, a cutaneous delivery device, a nebulizer, or an inhaler.

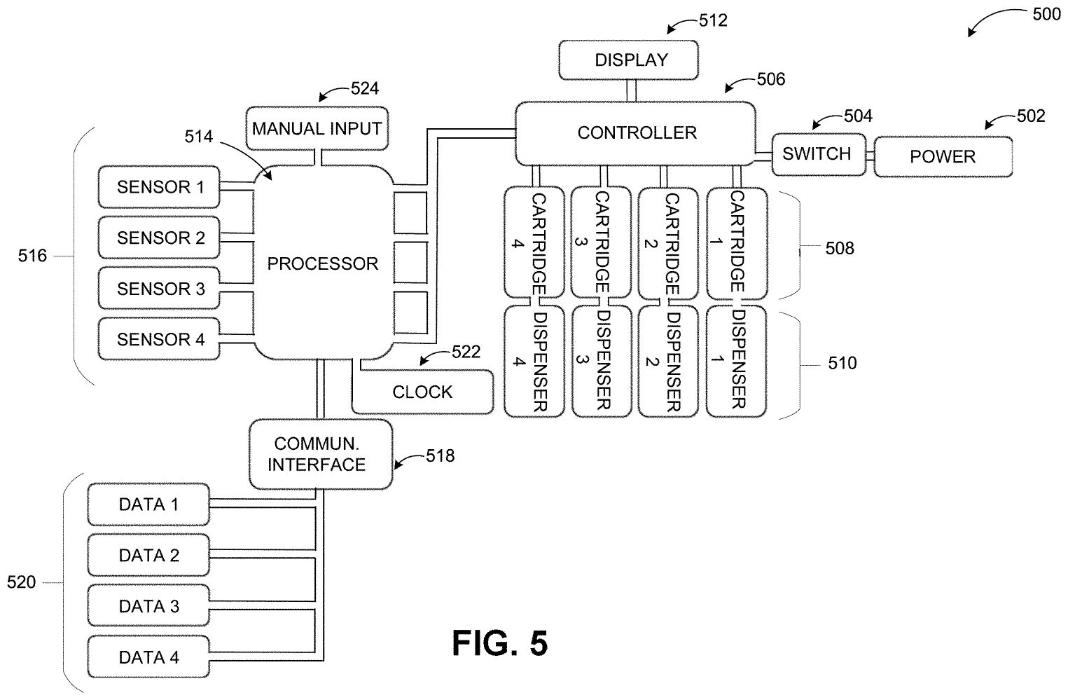

FIG. 5 includes a conceptual illustration of an example apparatus configured to control and optimize agent delivery to an individual in a tailored manner.

As shown in a diagram 500, the example apparatus may be a delivery device. The apparatus may include a processor 514 coupled to, among other things, a controller 506. The processor 514 may predefine a timing and a dosage of one or more agents to be delivered to an individual based on an average time and dosage standard for each agent. The average time and dosage standard may be based on previous testing or population statistics, for example. The processor 514 may then modify the average time and dosage standard based on characteristics of the individual, such as age, body mass index (i.e., height and weight), and sex, to obtain the predefined timing and dosage. The processor 514 may provide instructions to the controller 506 regarding the delivery of each agent to the individual at the respective predefined timing and dosage. The predefined timing and dosage may indicate a time of day for delivery, a frequency of delivery, a rate of delivery, a number of dosages to be delivered, and/or an actual amount or size of the agent delivered, among other examples.

The controller 506 may be coupled to cartridges 508 configured to hold the agents and dispensers 510 configured to, upon release from the cartridges 508, receive the agents and dispense the agents to the individual. The controller 506 may control the release of the agent from one or more of the cartridges 508 and the dispensing of the agent to the individual by one or more of the dispensers 510 based on the instructions provided by the processor 514. In some embodiments, the apparatus may include manual input control elements 524 to enable the individual to control the time and the dosage of the delivery of the agent. The apparatus may include a clock 522 coupled to the processor 514 to prompt delivery of the agent to the individual at an appropriate time based on the predefined timing.

The apparatus may include one or more sensors 516. The sensors 516 may include accelerometers, audio sensors such as microphones, visual sensors such as cameras and light sensors, gyroscopes, and/or biometric sensors as a thermometer, a heart rate monitor, an electrocardiogram, or electromyographic sensors, among other examples. Additionally, the apparatus may include a communication interface 518 that is configured to facilitate communication between the apparatus and one or more other sensors, computing devices, smart devices, and/or networks associated with the individual. The processor 514 may be configured to receive data 520 from the other sensors, computing devices, smart devices, and/or networks through the communication interface 518. The data 520 may include sensor data, similar to the input received from the sensors 516, in addition to application or network based data that may be associated with parameters related to or indicate events that may affect physiological and/or cognitive functions of the individual.

The processor 514 may analyze input from the sensors 516 and the data 520 to determine an efficacy of each agent. For example, prior to the delivery of the agents, input from the sensors 516 and the data 520 may be analyzed to define a baseline profile for the individual based on a first set of values for the parameters related to the physiological and/or cognitive functions of the individual. Following the delivery of the agents, new input from the sensors 516 and the data 520 may be received and analyzed to determine a second set of values for the parameters related to the physiological and/or cognitive functions of the individual. The second set of values may be compared to the first set of values that define the baseline profile in order to determine the efficacy of each agent. For example, if a first agent is a memory-enhancing agent, values for parameters related to memory-based cognitive functions may be compared between the first set and the second set to determine efficacy of the first agent. Similarly, if a second agent is a focus-enhancing agent, values for parameters related to focus-based cognitive functions may be compared between the first set and the second set to determine efficacy of the second agent. If the individual manually controls delivery of one or more of the agents through the manual input control elements 524, the processor 514 may receive data recorded by the controller 506 that includes a time and a dosage selected by the individual for the delivery of the agents, and may process the recorded data in conjunction with the input from the sensors 516 and the data 520 to analyze the efficacy of each agent. In addition to processing the input to analyze efficacy of each agent individually, the input may be further processed to determine interactions among the agents.

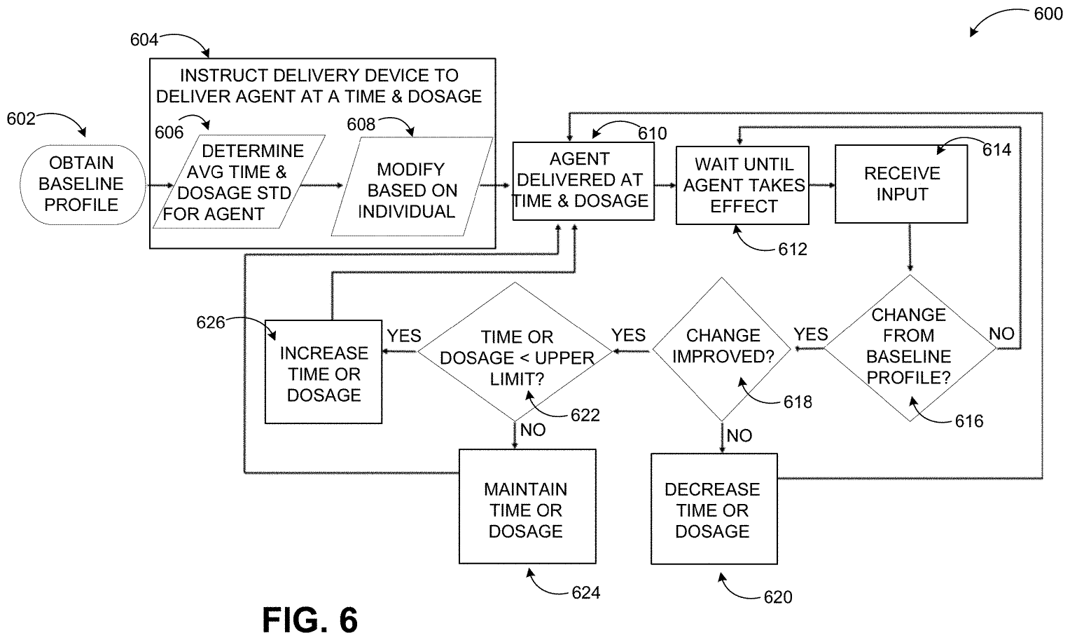

Based on the efficacy analysis of the agents, individually and in combination, the processor 514 may then adjust the predefined timing and/or dosage of the agent, as described in detail in FIG. 6 below. In some embodiments, additional data related to the efficacy of the agent with respect to a physiological function and/or a cognitive function of a plurality of individuals may be received. The predefined time and/dosage at which the agent is to be delivered to the individual may be further adjusted based on both the analysis and the data. For example, population level data from the plurality of individuals may further inform optimized time and dose scheduling from a larger data pool.

In some examples, the apparatus may include a display 512. The display 512 may present information associated with a timing and a dosage (e.g., the predefined timing and dosage or a later adjusted timing and dosage) at which the agent is delivered to the individual. For example, the display 512 may be an analog clock that visually represents a type and dosage of each agent delivered in conjunction with the time that the agent was or will be delivered. The display 512 may also present performance results based on the analysis of the efficacy of the agent.

Additionally, the apparatus may include a power supply 502 to enable operation of the delivery device. The power supply 502 may include a battery, such as rechargeable battery that may be remotely charged on an induction charger, for example. Alternatively, the power supply 502 may include a motion capture device, or a solar power converter, among other examples. In some embodiments, in response to one or more of the sensors 516 detecting a proximate position of the delivery device to the individual, the processor 514 may prompt the controller 506 to automatically activate the power supply 502 through a switch 504. Similarly, in response to one or more of the sensors 516 detecting a removal of the delivery device from the proximate position to the individual, the processor 514 may prompt the controller 506 to automatically deactivate the power supply 502 through the switch 504.

FIG. 6 includes a conceptual illustration of timing and dosage adjustment of an agent based on analysis of input retrieved prior to and following the delivery of the agent.

As shown in a diagram 600, a baseline profile of an individual may be obtained 602 prior to delivery of an agent to the individual. The baseline profile may be based on initial input received from sensors, computing devices, smart devices, and/or networks associated with the individual. For example, the initial input may include sensor and data feed associated with parameters related to physiological and/or cognitive functions of the individual, such as the individual's temperature, heart rate, heart electrical activity, muscle electrical activity, sleep and wake patterns, diet, movement patterns, reaction times, responsiveness, speed and error rates associated with tasks, and results of cognitive assessments, among other similar functions. The baseline profile may be defined for the individual based on a first set of values for the parameters received in the initial input. Additionally, the data feed may indicate events that may affect physiological and/or cognitive functions of the individual, such as planned sleep times, meetings where alertness or cognitive functioning is particularly important, exam times where stress levels may be elevated, exercise programs, or travel, among other examples. These events may be extracted from calendar or communication applications, as well as social or professional networks. Knowledge of these events may be used to proactively adjust a timing and a dosage of the agent for delivery.

A delivery device may be instructed to deliver the agent to the individual at a timing and dosage 604. The timing and dosage may be predefined based on a determined average time and dosage standard for the agent 606, which is further modified based on the individual 608. For example, the determined average time and dosage standard for the agent may be based on previous testing or population statistics, which may be modified based on the individual's age, body mass index (i.e., height and weight), sex, and/or other similar characteristics. The average time and dosage standard may also be modified based on knowledge of events associated with the individual that may affect physiological and/or cognitive functions of the individual, as described above. The timing and dosage may indicate a time of day for delivery, a frequency of delivery, a rate of delivery, a number of dosages to be delivered, and/or an actual amount or size of the agent delivered, among other examples. The agent may then be delivered to the individual at the instructed time and dosage 610, and a particular waiting period may be allowed to pass based on a type of the agent in order to allow the agent to take potential effect on the individual 612.

Following the agent's delivery and waiting period, input may be received 614. The input may include similar input to the initial input received prior to the agent's delivery, such as sensor and data feed associated with parameters related to or events that may affect physiological and/or cognitive functions of the individual, from the sensors, computing devices, smart devices, and networks associated with the individual. The input may be analyzed to determine whether there has been a change from the baseline profile 616 in order to analyze the efficacy of the agent. For example, a second set of values for the parameters received in the input may be compared to the baseline profile. More specifically, the second set of values for the parameters received in the input may be compared to the first set of values for the parameters received in the initial input used to define the baseline profile.

If the input analysis yields no change from the baseline profile, an additional waiting period may be allowed to pass to give the agent more time to take potential effect, and another input may be received and processed for analysis to determine whether there has been a change from the baseline profile. The analysis may include a comparison of a third set of values for the parameters received in the additional input to the first set of values for the parameters received in the initial input used to define the baseline profile. If the input analysis yields a change from the baseline profile, a determination is made as to whether the change indicates the individual's physiological and/or cognitive functions have improved 618. The time and/or dosage of the agent for a next delivery to the individual may then be adjusted based on the determination to optimize efficacy of the agent in a tailored manner. Adjustments may be made to the time of day of delivery, the frequency of delivery, the rate of delivery, the number of dosages delivered, and/or the actual amount or size of the agent delivered, among other similar adjustments.

For example, if the change does not indicate an improvement, the time and/or dosage for a next delivery of the agent may be decreased 620. If the change does indicate an improvement, a determination is made as to whether the time and/or dosage at which the agent was delivered is below the upper limit threshold for the type of the agent 622. If the time and/or dosage is at the upper limit threshold, the time and/or dosage may be maintained 624 for a next delivery of the agent. If the time and/or dosage is below the upper limit threshold, the time and/or dosage may be increased 626 for a next delivery of the agent.

The above-described operations of agent delivery, input processing and analysis, and time and/or dosage adjustment based on the analysis may be continuously repeated. Through this iterative process, the most effective time and dosage may be learned for an agent individually, and in combination with the one or more other agents that may be delivered to the individual in conjunction with the agent.

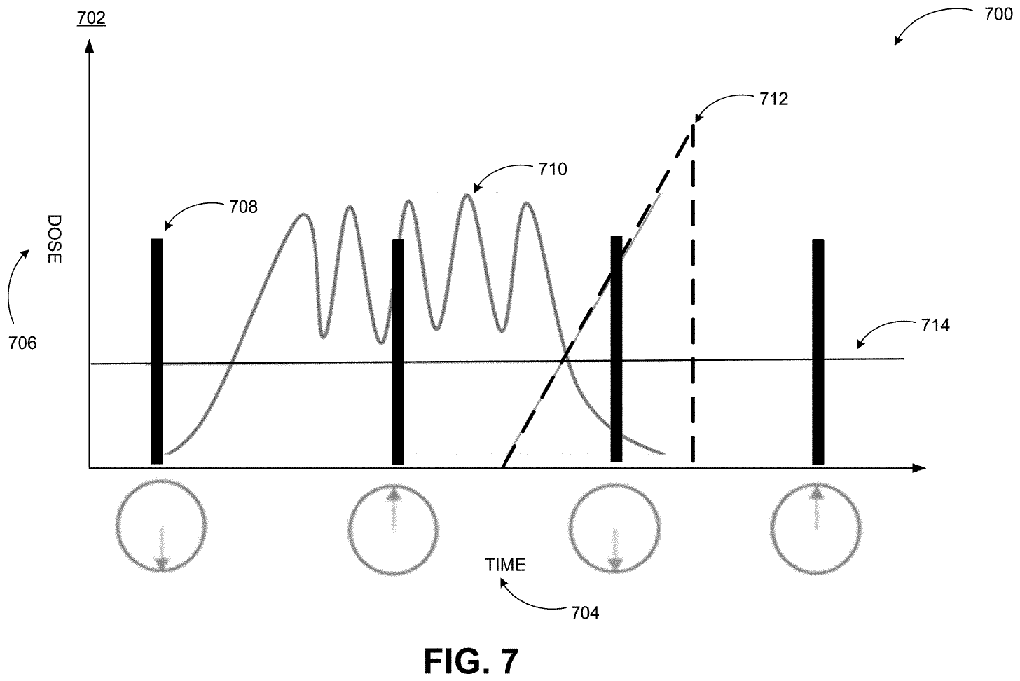

FIG. 7 includes a conceptual illustration of schedule variability for a given agent. The schedule for a given agent may have many different variables, such as the shape or patterns, timing, and regularity. In diagram 700, a graph 702, with an x-axis representing time 704 and a y-axis representing dose 706, illustrates example schedule profiles for an agent.

Example schedule profiles may include intermittent regular dosing 708, oscillatory dosing 710, ramp dosing 712, or continuous dosing 714. For example, if the agent requires intermittent regular dosing 708, a same dosage of the agent may be delivered to the individual intermittently at equal, periodic time intervals. If the agent requires oscillatory dosing 710, a dosage of the agent delivered to the individual may be iteratively increased and decreased in an oscillatory manner for a particular time period. If the agent requires ramp dosing 712, a dosage of the agent delivered to the individual may be gradually increased for a particular time period until the dosage reaches an upper limit threshold and the delivery is stopped. If the agent requires continuous dosing 714, a same dosage of the agent may be delivered continuously to individual throughout the day and night.

The example schedule profiles illustrated by the graph 702 are more typical example schedules, but there are many more, including non-regular patterns, such as what might happen when agents are delivered in response to an event, or the delivery is manually controlled by the individual. In many cases, a given agent may likely have a given subset of schedules that are appropriate. For example, delivery of motivation enhancers may be intermittent corresponding to lull points in the day, such as after meals, and memory enhancers may be oscillatory during REM sleep.

As discussed previously, nootropics and other similar regimens recommend an individual to take multiple different types of agents that individually target specific functions and in combination contribute to an overall physiological or cognitive improvement. This is often referred to as a full stack. In many cases, each of these agents may need to be taken at different dosages and at different times of the day. Currently, most of these agents are either in pill or powder form that are taken orally, and thus a full stack program typically requires a person to take multiple pills at different times of the day. The graph 702 demonstrates how timing and dosage for delivery of an agent may vary drastically based on a schedule of an agent, which further informs how difficult it would be for the individual to self-manage the conventional oral delivery of multiple agents (e.g., an individual on a full stack program) when each of these agents has varied timing and dosage standards.

Embodiments as described herein, relieve such difficulty and potential for error leading to inefficacy or harmful effects by automatically controlling the delivery of the multiple agents to the individual. Moreover, the embodiments provide individually-tailored optimization of the timing and dosage for the delivery of the agents based on sensor and data feed related to physiological and/or cognitive functions of the individual in order to increase an efficacy of the agent with respect to that individual.

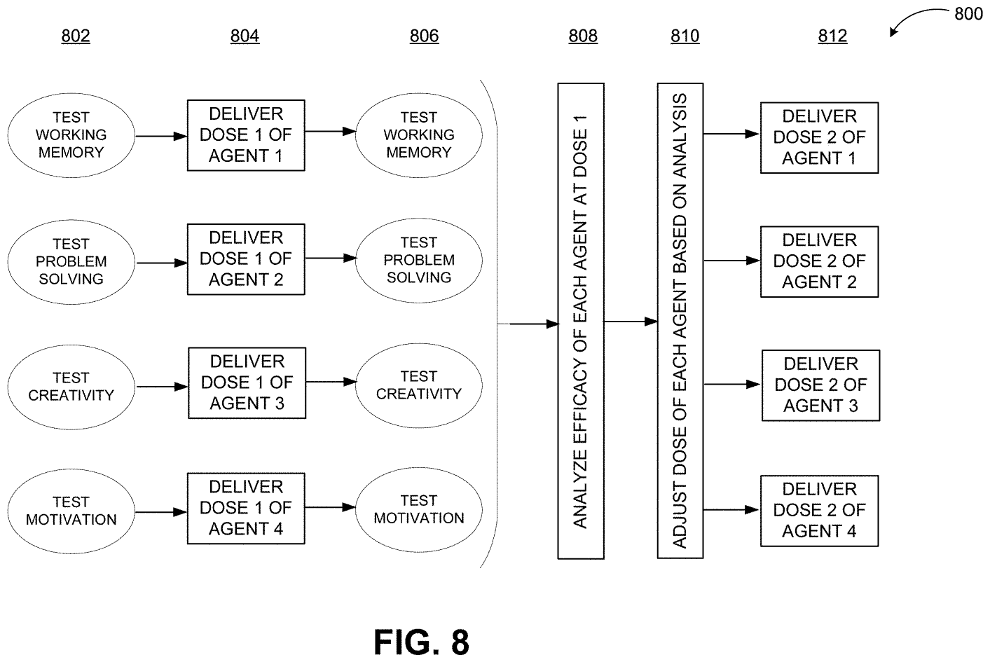

FIG. 8 includes a conceptual illustration of timing and dosage adjustment of multiple agents based on an efficacy analysis of the agents, individually and in combination. In an example embodiment, a nootropics full stack regimen may be comprised of multiple different types of agents individually targeting specific cognitive functions, such as working memory, problem solving, creativity, and motivation, that when taken together, effectively contribute to an individual's overall cognitive enhancement.

As shown in diagram 800, cognitive functions corresponding to agents within the stack that are configured to target those particular cognitive functions may be tested at operation 802 prior to delivering the agents to determine a baseline profile for the individual. For example, working memory corresponding to agent 1, problem solving corresponding to agent 2, creativity corresponding to agent 3, and motivation corresponding to agent 4 may be tested. To perform this testing, sensors, computing devices, smart devices, and/or networks associated with the individual may be utilized to obtain data related to these functions. In one example, a typing rate or an eye gaze of the individual measured via applications being executed on or sensors integrated with one or more of the individual's computing devices may provide data related to motivation. In another example, an application executing on one or more of the individual's computing devices may provide cognitive assessments to the individual to complete in order to obtain data related to problem solving, working memory, and/or creativity. The data obtained to define the baseline profile may represent a first set of values for parameters related to the functions of working memory, problem solving, creativity, and motivation.

The agents within the stack may be delivered to the individual at operation 804. A first dose of each agent and a timing at which the first dose is delivered may be based on an average time and dosage standard for a type of the agent, which may be further modified based on the individual's baseline profile, as well as characteristics of the individual, such as age, weight, and sex. Following delivery, the cognitive functions corresponding to the agents may be re-tested at operation 806 in order to obtain data related to these functions now that the agents have been delivered. The sensors, computing devices, smart devices, and/or networks associated with the individual may obtain data similar to that obtained in the initial testing performed at operation 802. For example, the data obtained may represent a second set of values for the parameters related to the functions of working memory, problem solving, creativity, and motivation.

The efficacy of each delivered agent at the first dose may then be analyzed at operation 808 based on this re-test data. To analyze efficacy, each value within the second set of values for the function-related parameters obtained after the agents were delivered may be compared to the corresponding value within the first set of values for the function-related parameters obtained prior to the delivery of the agents. If the comparison shows no change from the baseline for one or more of the functions, the processes as described in operations 806 and 808 may be repeated after waiting a particular time period to allow additional opportunity for the one or more agents corresponding to those one or more functions to take potential effect. If the comparison shows a change from the baseline, then the first dose at which each agent was delivered may be adjusted to a second dose for a next delivery based on the analysis at operation 810. For example, if the comparison shows functional improvement from the baseline, an efficacy of the agent may be inferred. Thus, the second dose may either be increased from the first dose, if the first dose is not above an upper limit threshold dosage for the agent, or may be maintained. Alternatively, if the comparison shows functional decline from the baseline, the second dose may be decreased from the first dose. In addition to analyzing efficacy of each agent individually, the input may be further processed to determine interactions among each of the agents, where the adjustments to the timing and/or dosage of the agents may be based on both the analysis and the determined interactions. In some embodiments, data related to the efficacy of the agent with respect to a physiological function and/or a cognitive function of a plurality of individuals may also be received. Any adjustments from the first dose to the second dose may be made based on both the analysis and the data. For example, population level data from the plurality of individuals may further inform optimized time and dose scheduling of the agents from a larger data pool.

The agents within the stack may then be delivered to the individual at the second dose at operation 812. The processes as described in operations 806 through 810 may then be repeated to further optimize the timing and/or dosage of each agent delivered to the individual.

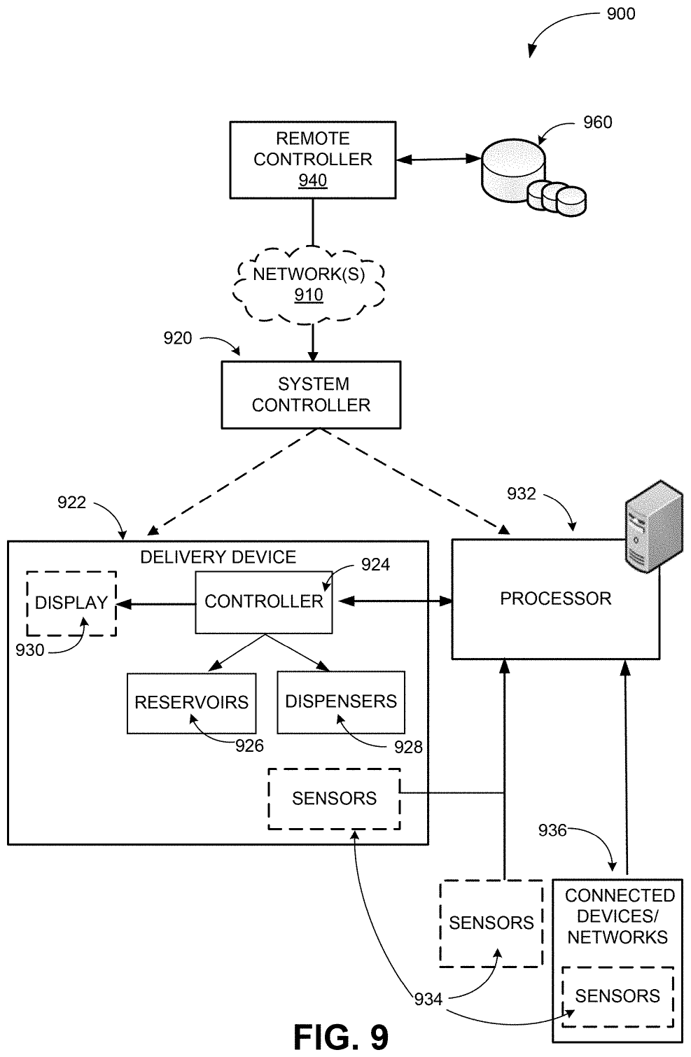

FIG. 9 illustrates major components of an example system configured to control and optimize delivery of agents to an individual in a tailored manner, arranged in accordance with at least some embodiments described herein.

As shown in diagram 900, a delivery device 922 configured to deliver an agent to an individual and a processor 932 configured to control the delivery of the agent to the individual may be governed by a system controller 920. The system controller 920 may be managed manually through a variety of inputs, may operate automatically after receiving one or more instructions, or may be operated independently by software. The system controller 920 may also be partially or entirely managed by a remote controller 940, for example, via network 910. The remote controller 940 may be managed manually through a variety of inputs, may operate automatically after receiving one or more instructions, or may be operated independently by software. Data associated with controlling the different processes for control and optimization of delivery of the agent to the individual may be stored at and/or received from data stores 960.

The delivery device 922 may include at least a controller 924, one or more reservoirs 926, and one or more dispensers 928. The reservoirs 926 may be configured to hold one or more agents, where the agents held by each reservoir or by each cartridge within a single reservoir may be a same type of agent at a same dosage, a same type of agent at a different dosage, a different type of agent at a same dosage, or a different type of agent at a different dosage. The dispensers 928 may be configured to receive the agents upon release from the reservoirs 926 and dispense the agents for delivery to the individual. Similar to the reservoirs 926, each dispenser may be configured to dispense a same type of agent at a same dosage, a same type of agent at a different dosage, a different type of agent at a same dosage, or a different type of agent at a different dosage. In some embodiments, each dispenser may correspond to a particular reservoir to prevent cross-contamination or mixing of different types of agents.

The controller 924 may be coupled to the reservoirs 926 and the dispensers 928 and configured to execute instructions for delivery of the agents to the individual at a predefined timing and dosage, where the instructions are received from the processor 932. For example, the controller 924 may prompt the release of the agents from the reservoirs 926 and the dispensing of the agents from the dispensers 928 at the predefined timing and dosage. In some embodiments, the delivery device 922 may include a display 930 that presents information associated with a timing and a dosage (e.g., the predefined timing and dosage) at which the agents are delivered to the individual. In some examples, the display 930 may include a clock.

As illustrated in diagram 900, the processor 932 may be separate from the delivery device 922. For example, the processor 932 may be a component of a computing device associated with the individual, such as a smart phone, a tablet, or a laptop. Alternatively, the processor 932 may be an integrated component of the delivery device 922. As previously discussed, the processor 932 may provide instructions to the controller 924 of the delivery device 922 for the delivery of the agents to the individual at a predefined time and dosage. Following, the delivery of the agents to the individual, the processor 932 may then receive input associated with parameters related to physiological and/or cognitive functions of the individual. This input may include sensor data and feed data received from one or more sensors 934 and other computing and smart devices/networks 936 coupled to the processor 932. The sensors 934 may be associated with the delivery device 922, the other computing and smart devices/networks 936, or a computing device comprising the processor 932. Alternatively, the sensors 934 may be standalone sensor devices communicatively connected to the processor 932.

The processor 932 may then process the input to analyze an efficacy of the agents with respect to the physiological and/or cognitive function of the individual, and adjust the predefined timing and/or dosage for delivery of the agents based on the analysis in order to optimize the efficacy of the agents. The adjusted timing and dosage may be sent in a form of instructions to the controller 924 for execution at the appropriate time. In some embodiments, physiological and cognitive performance results based on the efficacy analysis may be presented to the individual. The performance results may be provided for presentation through the display 930 of the delivery device 922. Alternatively, the performance results may be provided for presentation through a display of a computing device comprising the processor 932.

The examples provided in FIGS. 1 through 9 are illustrated with specific systems, devices, and processes. Embodiments are not limited to environments according to these examples. Individually tailored control and optimization of agent delivery may be implemented in environments employing fewer or additional systems, devices, and processes. Furthermore, the example systems, devices, and processes shown in FIGS. 1 through 9 may be implemented in a similar manner with other values using the principles described herein.

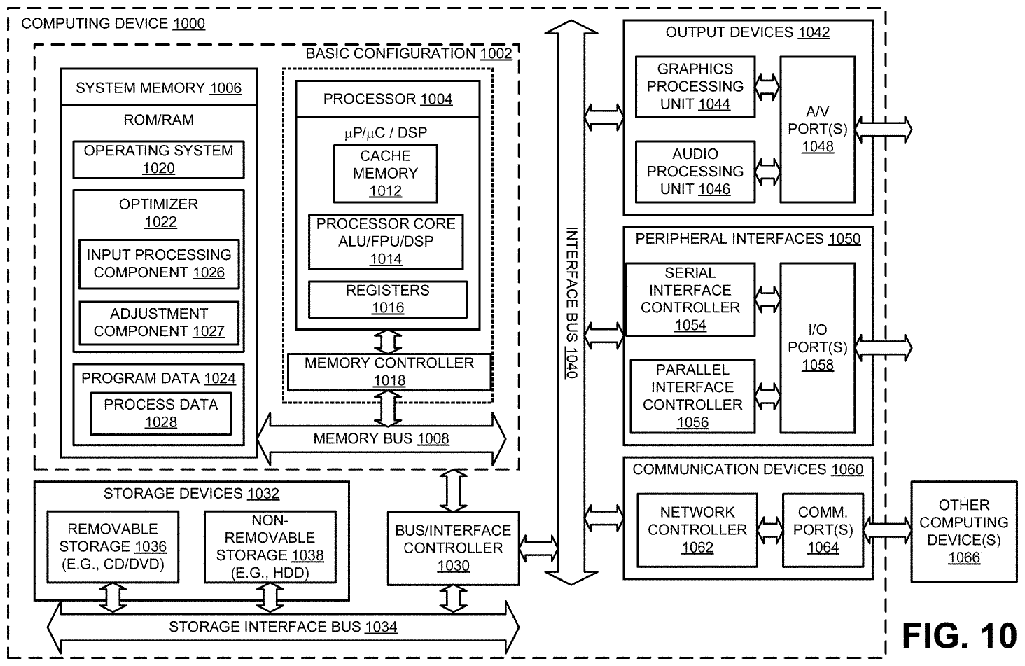

FIG. 10 illustrates a computing device, which may be used in conjunction with a delivery device to control and optimize delivery of agents to an individual in a tailored manner, arranged in accordance with at least some embodiments described herein.

In an example basic configuration 1002, the computing device 1000 may include one or more processors 1004 and a system memory 1006. A memory bus 1008 may be used to communicate between the processor 1004 and the system memory 1006. The basic configuration 1002 is illustrated in FIG. 10 by those components within the inner dashed line.

Depending on the desired configuration, the processor 1004 may be of any type, including but not limited to a microprocessor (μP), a microcontroller (μC), a digital signal processor (DSP), or any combination thereof. The processor 1004 may include one or more levels of caching, such as a cache memory 1012, a processor core 1014, and registers 1016. The example processor core 1014 may include an arithmetic logic unit (ALU), a floating point unit (FPU), a digital signal processing core (DSP core), or any combination thereof. An example memory controller 1018 may also be used with the processor 1004, or in some implementations, the memory controller 1018 may be an internal part of the processor 1004.

Depending on the desired configuration, the system memory 1006 may be of any type including but not limited to volatile memory (such as RANI), non-volatile memory (such as ROM, flash memory, etc.) or any combination thereof. The system memory 1006 may include an operating system 1020, an optimizer 1022, and program data 1024. The optimizer 1022 may include an input processing component 1026 and an adjustment component 1027. The optimizer 1022 may be configured to receive an input associated with parameters related to a physiological and/or cognitive function of the individual following the delivery of the agent to the individual at a predefined timing and dosage. The input processing component 1026 may be configured to process the input to analyze an efficacy of the agent with respect to the physiological and/or cognitive function of the individual. The adjustment component 1027 may be configured to adjust the predefined time and/or dosage for delivery of the agent to the individual based on the analysis. The program data 1024 may include process data 1028 such as an average time and dosage standard for the agent, characteristics of the individual, a baseline profile of the individual, input received at various stages prior to and following delivery of the agent, and upper limit thresholds for time and dosage of the agent, among other data, as described herein.

The computing device 1000 may have additional features or functionality, and additional interfaces to facilitate communications between the basic configuration 1002 and any desired devices and interfaces. For example, a bus/interface controller 1030 may be used to facilitate communications between the basic configuration 1002 and one or more data storage devices 1032 via a storage interface bus 1034. The data storage devices 1032 may be one or more removable storage devices 1036, one or more non-removable storage devices 1038, or a combination thereof. Examples of the removable storage and the non-removable storage devices include magnetic disk devices such as flexible disk drives and hard-disk drives (HDDs), optical disk drives such as compact disc (CD) drives or digital versatile disk (DVD) drives, solid state drives (SSDs), and tape drives to name a few. Example computer storage media may include volatile and nonvolatile, removable and non-removable media implemented in any method or technology for storage of information, such as computer readable instructions, data structures, program modules, or other data.

The system memory 1006, the removable storage devices 1036 and the non-removable storage devices 1038 are examples of computer storage media. Computer storage media includes, but is not limited to, RAM, ROM, EEPROM, flash memory or other memory technology, CD-ROM, digital versatile disks (DVDs), solid state drives (SSDs), or other optical storage, magnetic cassettes, magnetic tape, magnetic disk storage or other magnetic storage devices, or any other medium which may be used to store the desired information and which may be accessed by the computing device 1000. Any such computer storage media may be part of the computing device 1000.

The computing device 1000 may also include an interface bus 1040 for facilitating communication from various interface devices (e.g., one or more output devices 1042, one or more peripheral interfaces 1050, and one or more communication devices 1060) to the basic configuration 1002 via the bus/interface controller 1030. Some of the example output devices 1042 include a graphics processing unit 1044 and an audio processing unit 1046, which may be configured to communicate to various external devices such as a display or speakers via one or more A/V ports 1048. One or more example peripheral interfaces 1050 may include a serial interface controller 1054 or a parallel interface controller 1056, which may be configured to communicate with external devices such as input devices (e.g., keyboard, mouse, pen, voice input device, touch input device, etc.) or other peripheral devices (e.g., printer, scanner, etc.) via one or more I/O ports 1058. An example communication device 1060 includes a network controller 1062, which may be arranged to facilitate communications with one or more other computing devices 1066 over a network communication link via one or more communication ports 1064. The one or more other computing devices 1066 may include servers at a datacenter, customer equipment, and comparable devices.

The network communication link may be one example of a communication media. Communication media may be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

The computing device 1000 may be implemented as a part of a specialized server, mainframe, or similar computer that includes any of the above functions. The computing device 1000 may also be implemented as a personal computer including both laptop computer and non-laptop computer configurations.

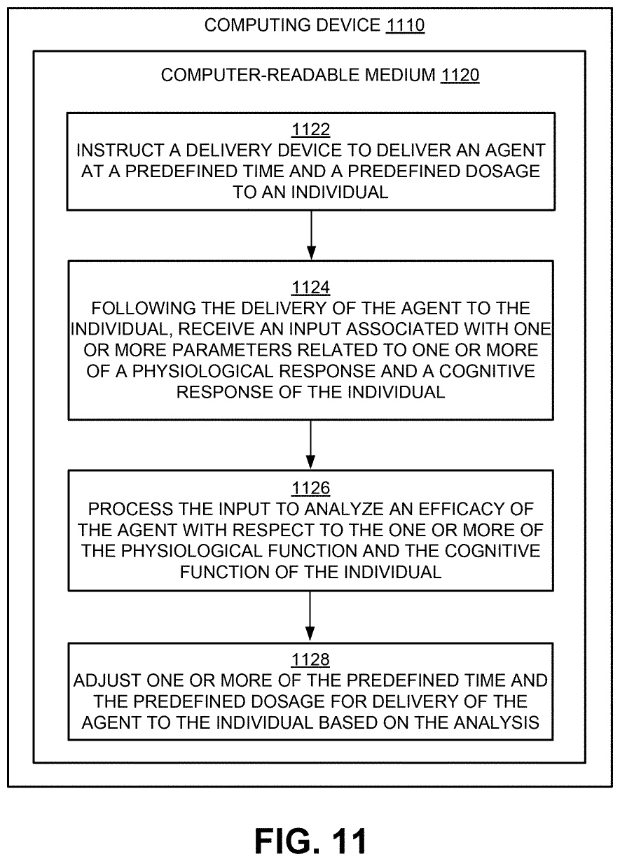

FIG. 11 is a flow diagram illustrating an example method to control and optimize delivery of agents to an individual in a tailored manner performed by a computing device such as the computing device in FIG. 10 in conjunction with a delivery device such as the delivery device described in FIG. 1, arranged in accordance with at least some embodiments described herein.

Example methods may include one or more operations, functions, or actions as illustrated by one or more of blocks 1122, 1124, 1126, and 1128 may in some embodiments be performed by a computing device such as the computing device 1000 in FIG. 10. Such operations, functions, or actions in FIG. 10 and in the other figures, in some embodiments, may be combined, eliminated, modified, and/or supplemented with other operations, functions or actions, and need not necessarily be performed in the exact sequence as shown. The operations described in the blocks 1122-1128 may be implemented through execution of computer-executable instructions stored in a computer-readable medium such as a computer-readable medium 1120 of a computing device 1110.

An example process to control delivery of an agent may begin with block 1122, “INSTRUCT A DELIVERY DEVICE TO DELIVER AN AGENT AT A PREDEFINED TIME AND A PREDEFINED DOSAGE TO AN INDIVIDUAL”, where the predefined time and dosage may be obtained by determining an average time and dosage standard for a type of the agent and modifying the standard based on the individual. The type of the agent may be a drug, a supplement, or a cognitive-enhancing substance, such as a nootropic.

Block 1122 may be followed by block 1124, “FOLLOWING THE DELIVERY OF THE AGENT TO THE INDIVIDUAL, RECEIVE AN INPUT ASSOCIATED WITH ONE OR MORE PARAMETERS RELATED TO ONE OR MORE OF A PHYSIOLOGICAL RESPONSE AND A COGNITIVE RESPONSE OF THE INDIVIDUAL”, where the input includes the individual's temperature, heart rate, heart electrical activity, muscle electrical activity, sleep and wake patterns, diet, movement patterns, reaction times, responsiveness, speed and error rates associated with tasks, and/or results of cognitive assessments. The input may be obtained from the computing device 1110 itself and/or received from the delivery device or other computing devices, smart devices, and networks communicatively coupled to the computing device 1110.

Block 1124 may be followed by block 1126, “PROCESS THE INPUT TO ANALYZE AN EFFICACY OF THE AGENT WITH RESPECT TO THE ONE OR MORE OF THE PHYSIOLOGICAL FUNCTION AND THE COGNITIVE FUNCTION OF THE INDIVIDUAL”, where prior to the delivery of the agent to the individual, a baseline profile may be defined for the individual based on a first set of values for the parameters related to the physiological and/or the cognitive function of the individual received in an initial input. The input received following the delivery of the agent to the individual may be processed and the efficacy of the agent may be analyzed by comparing a second set of values for the parameters received in the input to the baseline profile.

Block 1126 may be followed by block 1128, “ADJUST ONE OR MORE OF THE PREDEFINED TIME AND THE PREDEFINED DOSAGE FOR DELIVERY OF THE AGENT TO THE INDIVIDUAL BASED ON THE ANALYSIS”, where the analysis may lead to four possible adjustment scenarios. First, a predefined time and/or dosage may be decreased if the second set of values indicate a decline of the physiological function and/or the cognitive function of the individual from the baseline profile. Second, the predefined time and/or dosage may be increased if the second set of values indicate an improvement of the physiological and/or cognitive function of the individual from the baseline profile and the predefined time and/or dosage is determined to be below the upper limit threshold for the type of the agent. Third, the predefined time and/or dosage may be maintained if the second set of values indicate an improvement of the physiological and/or cognitive function of the individual from the baseline profile, but the predefined time and/or dosage is determined to be at the upper limit threshold for the type of the agent. Fourth, if the second set of values indicate no change of the physiological and/or cognitive function of the individual from the baseline profile, another input associated with the parameters may be received after waiting a particular period of time, and a third set of values for the parameters received in the other input may be compared to the baseline profile to analyze the efficacy of the agent. This analysis may similarly lead to the four possible adjustment scenarios described above.

The operations included in process 1100 are for illustration purposes. Control and optimization of agent delivery may be implemented by similar processes with fewer or additional operations, as well as in different order of operations using the principles described herein. The operations described herein may be executed by one or more processors operated on one or more computing devices, one or more processor cores, and/or specialized processing devices, among other examples.

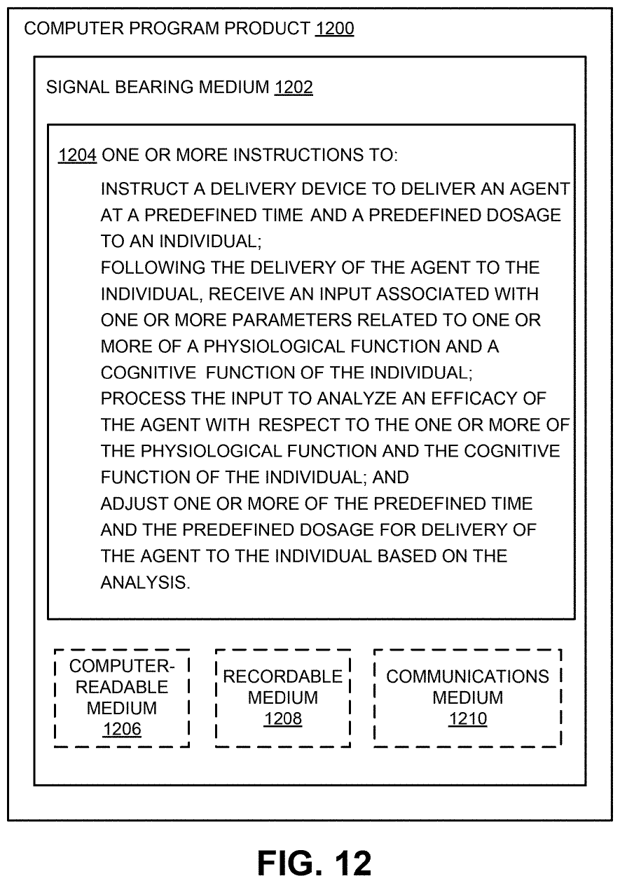

FIG. 12 illustrates a block diagram of an example computer program product, arranged in accordance with at least some embodiments described herein.

In some examples, as shown in FIG. 12, a computer program product 1200 may include a signal bearing medium 1202 that may also include one or more machine readable instructions 1204 that, in response to execution by, for example, a processor may provide the functionality described herein. Thus, for example, referring to the processor 1004 in FIG. 10, the optimizer 1022 may perform or control performance of one or more of the tasks shown in FIG. 12 in response to the instructions 1204 conveyed to the processor 1004 by the signal bearing medium 1202 to perform actions associated with the control and optimization of agent delivery as described herein. Some of those instructions may include, for example, instruct a delivery device to deliver an agent at a predefined time and a predefined dosage to an individual; following the delivery of the agent to the individual, receive an input associated with one or more parameters related to one or more of a physiological function and a cognitive function of the individual; process the input to analyze an efficacy of the agent with respect to the one or more of the physiological function and the cognitive function of the individual; and adjust one or more of the predefined time and the predefined dosage for delivery of the agent to the individual based on the analysis, according to some embodiments described herein.

In some implementations, the signal bearing medium 1202 depicted in FIG. 12 may encompass computer-readable medium 1206, such as, but not limited to, a hard disk drive (HDD), a solid state drive (SSD), a compact disc (CD), a digital versatile disk (DVD), a digital tape, memory, etc. In some implementations, the signal bearing medium 1202 may encompass recordable medium 1208, such as, but not limited to, memory, read/write (R/W) CDs, R/W DVDs, etc. In some implementations, the signal bearing medium 1202 may encompass communications medium 1210, such as, but not limited to, a digital and/or an analog communication medium (e.g., a fiber optic cable, a waveguide, a wired communication link, a wireless communication link, etc.). Thus, for example, the computer program product 1200 may be conveyed to one or more modules of the processor 1004 by an RF signal bearing medium, where the signal bearing medium 1202 is conveyed by the communications medium 1210 (e.g., a wireless communications medium conforming with the IEEE 802.11 standard).

According to some examples, a means to control delivery of agents may be provided. Example means may include instructing a delivery device to deliver an agent at a predefined time and a predefined dosage to an individual, following the delivery of the agent to the individual, receiving an input associated with one or more parameters related to a physiological function and/or a cognitive function of the individual, processing the input to analyze an efficacy of the agent with respect to the physiological function and/or the cognitive function of the individual, and adjusting the predefined time and/or the predefined dosage for delivery of the agent to the individual based on the analysis.

According to some embodiments, methods to control delivery of agents may be provided. An example method includes instructing a delivery device to deliver an agent at a predefined time and a predefined dosage to an individual, following the delivery of the agent to the individual, receiving an input associated with one or more parameters related to a physiological function and/or a cognitive function of the individual, processing the input to analyze an efficacy of the agent with respect to the physiological function and/or the cognitive function of the individual, and adjusting the predefined time and/or the predefined dosage for delivery of the agent to the individual based on the analysis.

In other embodiments, an average time and dosage standard may be determined for a type of the agent, and the average time and dosage standard may be modified based on characteristics of the individual to obtain the predefined time and the predefined dosage. Prior to the delivery of the agent to the individual, an initial input associated with the parameters related to the physiological function and/or the cognitive function of the individual may be received. A baseline profile may be defined for the individual based on a first set of values for the parameters received in the initial input prior to the delivery of the agent to the individual. A second set of values for the parameters received in the input following the delivery of the agent to the individual may be compared to the baseline profile to analyze the efficacy of the agent with respect to the physiological function and/or the cognitive function of the individual.

In further embodiments, response to a determination that the second set of values indicate a decline of the physiological function and/or the cognitive function of the individual from the baseline profile, the predefined time and/or the predefined dosage for delivery of the agent to the individual may be decreased. In response to a determination that the second set of values indicate an improvement of the physiological function and/or the cognitive function of the individual from the baseline profile, whether the predefined time and/or the predefined dosage is below an upper limit threshold for a type of the agent is determined. If the predefined time and/or the predefined dosage is below the upper limit threshold, the predefined time and/or the predefined dosage for delivery of the agent to the individual may be increased; else the predefined time and the predefined dosage for delivery of the agent to the individual may be maintained. In response to a determination that the second set of values indicate no change of the physiological function and/or the cognitive function of the individual from the baseline profile, another input associated with the parameters related to the physiological function and/or the cognitive function of the individual may be received after a particular period of time, and a third set of values for the received in the other input after the particular period of time may be compared to the baseline profile.

In yet further embodiments, data related to the efficacy of the agent with respect to a physiological function and/or a cognitive function of a plurality of individuals may be received, the predefined time and/or the predefined dosage at which the agent is to be delivered to the individual may be adjusted based on the analysis and the data. Data related to an environment of the individual may be received, a determination may be made as to whether the environment-related data indicates the physiological function and/or the cognitive function of the individual is affected, and the predefined time and/or the predefined dosage at which the agent is to be delivered to the individual may be further adjusted based on the environment-related data. The delivery device may be instructed to deliver the agent and another agent to the individual at respective predefined times and predefined dosages, the input may be processed to analyze an efficacy of the agent and the other agent with respect to the physiological function and/or the cognitive function of the individual and determine one or more interactions between the agent and the other agent, and one or more of the respective predefined times and predefined dosages for delivery of the agent and the other agent to the individual may be adjusted based on the analysis and the determined interactions. Performance results of the agent may be determined based on the analysis of the efficacy of the agent with respect to the physiological function and/or the cognitive function of the individual, and the performance results may be provided to be displayed to the individual.

According to some examples, computing devices configured to control delivery of agents may be described. An example computing device may include a memory configured to store instructions, and a processor coupled to the memory. The processor in conjunction with the instructions stored on the memory may be configured to instruct a delivery device to deliver an agent at a predefined time and a predefined dosage to an individual, following the delivery of the agent to the individual, receive an input associated with one or more parameters related to a physiological function and/or a cognitive function of the individual, process the input to analyze an efficacy of the agent with respect to the physiological function and/or the cognitive function of the individual, and adjust the predefined time and/or the predefined dosage for delivery of the agent to the individual based on the analysis.

In other examples, an average time and dosage standard for a type of the agent may be determined, and the average time and dosage standard may be modified based on characteristics of the individual to obtain the predefined time and the predefined dosage. The input may include the individual's temperature, heart rate, heart electrical activity, muscle electrical activity, sleep and wake patterns, diet, movement patterns, reaction times, responsiveness, speed and error rates associated with tasks, and/or results of cognitive assessments. The input may be received from sensors, other computing devices, and/or networks communicatively connected to the computing device over a wired or a wireless network. The sensors may include accelerometers, audio sensors, visual sensors, gyroscopes, and/or biometric sensors. The sensors may be components of the delivery device, components of the computing device, components of the devices communicatively connected to the computing device, or separate devices. The devices and networks associated with the individual may include wearable computing devices, mobile devices, smart home devices, smart transportation devices, and an internet of things.

In further examples, prior to the delivery of the agent to the individual, an initial input associated with the parameters related to the physiological function and/or the cognitive function of the individual may be received, a baseline profile may be defined for the individual based on a first set of values for the parameters received in the initial input prior to the delivery of the agent to the individual, and a second set of values for the parameters received in the input following the delivery of the agent to the individual may be compared to the baseline profile. The predefined time and/or the predefined dosage may be decreased in response to a determination that the second set of values indicate a decline of the physiological function and/or the cognitive function of the individual from the baseline profile, may be maintained or increased in response to a determination that the second set of values indicate an improvement of the physiological function and/or the cognitive function of the individual from the baseline profile, and in response to a determination that the second set of values indicate no change of the physiological function and/or the cognitive function of the individual from the baseline profile, another input associated with the parameters related to the physiological function and/or the cognitive function of the individual may be received after a particular period of time, where a third set of values for the parameters received in the other input after the particular period of time may be compared to the baseline profile.

In yet further examples, data related to the efficacy of the agent with respect to a physiological function and/or a cognitive function of a plurality of individuals may be received. The predefined time and/or the predefined dosage at which the agent is to be delivered to the individual may be adjusted based on the analysis and the data.

According to some embodiments, systems configured to deliver agents may be described. An example system may include a delivery device configured to deliver an agent to an individual and a computing device coupled to the delivery device and configured to control the delivery of the agent by the delivery device. An example delivery device may include a reservoir configured to hold the agent, a dispenser configured to receive the agent upon release from the reservoir and dispense the agent for delivery to the individual, and a controller coupled to the reservoir and the dispenser and configured to execute instructions for delivery of the agent to the individual at a predefined time and a predefined dosage. An example computing device may include a memory configured to store instructions and a processor coupled to the memory. The processor in conjunction with the instructions may be configured to provide the instructions for delivery of the agent to the individual at the predefined time and the predefined dosage to the controller, following the delivery of the agent to the individual, receive an input associated with one or more parameters related to a physiological function and/or a cognitive function of the individual, process the input to analyze an efficacy of the agent with respect to the physiological function and/or the cognitive function of the individual, and adjust the predefined time and/or the predefined dosage for delivery of the agent to the individual based on the analysis.

In other embodiments, the computing device may be integrated with the delivery device in a single device. The computing device may be a separate device that is communicatively connected to the delivery device over a wired or wireless network. An average time and dosage standard for a type of the agent may be determined, and the average time and dosage standard may be modified based on characteristics of the individual to obtain the predefined time and the predefined dosage.

In further embodiments, prior to the delivery of the agent to the individual, an initial input associated with the parameters related to the physiological function and/or the cognitive function of the individual may be received, a baseline profile may be defined for the individual based on a first set of values for the parameters received in the initial input prior to the delivery of the agent to the individual, and a second set of values for the parameters received in the input following the delivery of the agent to the individual may be compared to the baseline profile. The predefined time and/or the predefined dosage may be decreased in response to a determination that the second set of values indicate a decline of the physiological function and/or the cognitive function of the individual from the baseline profile, maintained or increased in response to a determination that the second set of values indicate an improvement of the physiological function and/or the cognitive function of the individual from the baseline profile, and, in response to a determination that the second set of values indicate no change of the physiological function and/or the cognitive function of the individual from the baseline profile, another input associated with parameters related to the physiological function and/or the cognitive function of the individual may be received after a particular period of time, where a third set of values for the parameters received in the other input after the particular period of time may be compared to the baseline profile.

In yet further embodiments, performance results of the agent may be determined based on the analysis of the efficacy of the agent with respect to the physiological function and/or the cognitive function of the individual. The performance results may be provided for display to the delivery device and/or one or more devices communicatively connected to the computing device over a wired or wireless network. The delivery device may also include a display configured to present information associated with the predefined time and the predefined dosage and the performance results. The delivery device may further include a clock coupled to the controller to prompt the delivery of the agent at the predefined time or an adjusted predefined time. The delivery device may yet further include control elements to enable the individual to manually control delivery of the agent. In response to the individual selecting to manually control delivery of the agent through the control elements of the delivery device, data recorded by the controller that includes a time and a dosage selected by the individual for the delivery of the agent may be recorded. The input and the recorded data may be processed to analyze an efficacy of the agent with respect to the physiological function and/or the cognitive function of the individual.

In some embodiments, the reservoir of the delivery device may include at least two cartridges or the delivery device may include at least two reservoirs. Each cartridge and/or each reservoir may be configured to hold the agent and at least another agent, respectively. The delivery device may be instructed to deliver the agent and the at least one other agent to the individual at respective predefined times and predefined dosages, the input may be processed to analyze an efficacy of each of the agents with respect to the physiological function and/or the cognitive function of the individual and determine one or more interactions between the agent and the other agent, and one or more of the respective predefined times and predefined dosages for delivery of the agent and the at least one other agent to the individual may be adjusted based on the analysis and the determined interactions.

In other embodiments, the delivery device may be a transdermal delivery device, an oral delivery device, a nasal delivery device, a sublingual delivery device, a buccal delivery device, an ocular delivery device, an otic delivery device, a rectal delivery device, a vaginal delivery device, an intravenous delivery device, an intramuscular delivery device, a subcutaneous delivery device, an intradermal delivery device, an intrathecal delivery device, a cutaneous delivery device, a nebulizer, and/or an inhaler. The transdermal delivery device may be a wearable computing device configured to contact a portion of skin of the individual when worn by the individual, a device configured to self-adhere to a portion of skin of the individual, or a device integrated in a clothing item that is configured to contact a portion of skin of the individual when the clothing item is worn by the individual. The agent may be a drug, a supplement, or a cognitive-enhancing substance. The agent may be a nootropic.

According to some examples, delivery devices configured to deliver agents may be described. An example delivery device may include a reservoir configured to hold an agent, a dispenser configured to receive the agent upon release from the reservoir and dispense the agent for delivery to an individual, and a controller coupled to the reservoir and the dispenser and configured to execute instructions received from a processor to deliver the agent to the individual at a predefined time and a predefined dosage. The predefined time and/or the predefined dosage may be adjusted based on an analysis of an efficacy of the agent with respect to a physiological function and/or a cognitive function of the individual performed by the processor.

In other examples, the processor may be a component of the delivery device or a computing device communicatively connected to the delivery device over a wired or a wireless network. The delivery device may include a display configured to present information associated with the predefined time and the predefined dosage and performance results of the agent based on the analysis of the efficacy of the agent with respect to the physiological function and/or the cognitive function of the individual received from the processor. The delivery device may also include sensors configured to provide input associated with one or more parameters related to the physiological function and/or the cognitive function of the individual to the processor. The sensors may include accelerometers, audio sensors, visual sensors, gyroscopes, and/or biometric sensors.

In further examples, the delivery device may be a transdermal delivery device. The transdermal delivery device may be a wearable computing device configured to contact a portion of skin of the individual when worn by the individual. The transdermal delivery device may be a device configured to self-adhere to a portion of skin of the individual. The transdermal delivery device may be a device integrated in a clothing item that is configured to contact a portion of skin of the individual when the clothing item is worn by the individual. The delivery device is may be an oral delivery device, a nasal delivery device, a sublingual delivery device, a buccal delivery device, an ocular delivery device, an otic delivery device, a rectal delivery device, a vaginal delivery device, an intravenous delivery device, an intramuscular delivery device, a subcutaneous delivery device, an intradermal delivery device, an intrathecal delivery device, a cutaneous delivery device, a nebulizer, and/or an inhaler.

In yet further examples, the reservoir may be refillable. The reservoir may include one or more cartridges configured to hold the agent, and the cartridges may be replaceable. The reservoir or the cartridges may comprise a level sensor configured to alert the controller when an amount of the agent remaining in the reservoir or cartridges is below a predefined threshold. The delivery device may include more than one reservoir. Each reservoir may be configured to hold one or more of a different type of agent and a different dosage of agent. The dispenser may be configured to dispense the agent onto a surface of a portion of skin of the individual or a substrate positioned on the surface of the portion of skin of the individual. The delivery device may include more than one dispenser. Each dispenser is configured to dispense one or more of a different type of agent and a different dosage of agent.

In some examples, the delivery device may include a power supply to enable operation of the delivery device. The power supply may include a battery, a motion capture device, and/or a solar power converter. The controller may be configured to automatically activate the power supply in response to detecting a proximate position of the delivery device to the individual and automatically deactivate the power supply in response to detecting a removal of the delivery device from the proximate position to the individual. The delivery device may include a clock coupled to the controller to prompt the delivery of the agent at the time or an adjusted time. The delivery device may include control elements to enable the individual to manually control the time and the dosage of the delivery of the agent. The agent may be a drug, a supplement, or a cognitive-enhancing substance. The agent may be a nootropic.

There are various vehicles by which processes and/or systems and/or other technologies described herein may be affected (e.g., hardware, software, and/or firmware), and the preferred vehicle will vary with the context in which the processes and/or systems and/or other technologies are deployed. For example, if an implementer determines that speed and accuracy are paramount, the implementer may opt for mainly hardware and/or firmware vehicle; if flexibility is paramount, the implementer may opt for mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, each function and/or operation within such block diagrams, flowcharts, or examples may be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, t some aspects of the embodiments disclosed herein, in whole or in part, may be equivalently implemented in integrated circuits, as one or more computer programs executing on one or more computers (e.g., as one or more programs executing on one or more computer systems), as one or more programs executing on one or more processors (e.g., as one or more programs executing on one or more microprocessors), as firmware, or as virtually any combination thereof, and that designing the circuitry and/or writing the code for the software and/or firmware are possible in light of this disclosure.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled. The terminology used herein is for the purpose of describing particular embodiments only, and is not intended to be limiting.

In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive (HDD), a compact disc (CD), a digital versatile disk (DVD), a digital tape, a computer memory, a solid state drive (SSD), etc.; and a transmission type medium such as a digital and/or an analog communication medium (e.g., a fiber optic cable, a waveguide, a wired communication link, a wireless communication link, etc.).

It is common within the art to describe devices and/or processes in the fashion set forth herein, and thereafter use engineering practices to integrate such described devices and/or processes into data processing systems. That is, at least a portion of the devices and/or processes described herein may be integrated into a data processing system via a reasonable amount of experimentation. A data processing system may include one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors.

A data processing system may be implemented utilizing any suitable commercially available components, such as those found in data computing/communication and/or network computing/communication systems. The herein described subject matter sometimes illustrates different components contained within, or connected with, different other components. Such depicted architectures are merely exemplary, and in fact, many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

For any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments are possible. The various aspects and embodiments disclosed herein are for purposes of illustration and are not intended to be limiting, with the true scope and spirit being indicated by the following claims.

1. A method to control delivery of agents, the method comprising:
instructing a delivery device to deliver an agent at a predefined time and a predefined dosage to an individual;
following the delivery of the agent to the individual, receiving an input associated with one or more parameters related to a cognitive function of the individual;
processing the input to analyze an efficacy of the agent with respect to the cognitive function of the individual; and
adjusting one or more of the predefined time and the predefined dosage for delivery of the agent to the individual based on the analysis.

2. The method of claim 1, wherein instructing the delivery device to deliver the agent at the predefined time and the predefined dosage to the individual comprises:
determining an average time and dosage standard for a type of the agent;
modifying the average time and dosage standard based on characteristics of the individual to obtain the predefined time and the predefined dosage; and
prior to the delivery of the agent to the individual, receiving an initial input associated with the one or more parameters related to the cognitive function of the individual.

3. (canceled)

4. The method of claim 2, further comprising:
defining a baseline profile for the individual based on a first set of values for the one or more parameters received in the initial input prior to the delivery of the agent to the individual; and
comparing a second set of values for the one or more parameters received in the input following the delivery of the agent to the individual to the baseline profile.

5. (canceled)

6. The method of claim 4, wherein adjusting one or more of the predefined time and the predefined dosage for delivery of the agent to the individual based on the analysis comprises:
in response to a determination that the second set of values indicate a decline of the cognitive function of the individual from the baseline profile, decreasing one or more of the predefined time and the predefined dosage for delivery of the agent to the individual;
in response to a determination that the second set of values indicate an improvement of the cognitive function of the individual from the baseline profile, determining whether one or more of the predefined time and the predefined dosage is below an upper limit threshold for a type of the agent; and
if the one or more of the predefined time and the predefined dosage is below the upper limit threshold, increasing one or more of the predefined time and the predefined dosage for delivery of the agent to the individual; else
maintaining the predefined time and the predefined dosage for delivery of the agent to the individual.

7. (canceled)

8. The method of claim 4, further comprising:
in response to a determination that the second set of values indicate no change of the cognitive function of the individual from the baseline profile:
after a particular period of time, receiving another input associated with the one or more parameters related to the one or more of the physiological function and the cognitive function of the individual; and
comparing a third set of values for the one or more parameters received in the other input after the particular period of time to the baseline profile.

9. The method of claim 1, further comprising:
receiving data related to an environment of the individual;
determining whether the environment-related data indicates the cognitive function of the individual is affected; and
adjusting the one or more of the predefined time and the predefined dosage at which the agent is to be delivered to the individual based on the analysis and the environment-related data.

10.-21. (canceled)

22. A system configured to deliver agents, the system comprising:
a delivery device configured to deliver an agent to an individual, the delivery device comprising:
a reservoir configured to hold the agent;
a dispenser configured to receive the agent upon release from the reservoir and dispense the agent for delivery to the individual; and
a controller coupled to the reservoir and the dispenser and configured to execute instructions for delivery of the agent to the individual at a predefined time and a predefined dosage; and
a computing device coupled to the delivery device and configured to control the delivery of the agent by the delivery device, the computing device comprising;
a memory configured to store instructions; and
a processor coupled to the memory, wherein the processor in conjunction with the instructions is configured to:
provide the instructions for delivery of the agent to the individual at the predefined time and the predefined dosage to the controller;
following the delivery of the agent to the individual, receive an input associated with one or more parameters related to a cognitive function of the individual;
process the input to analyze an efficacy of the agent with respect to the cognitive function of the individual; and
adjust one or more of the predefined time and the predefined dosage for delivery of the agent to the individual based on the analysis.

23. The system of claim 22, wherein the computing device is integrated with the delivery device in a single device or a separate device that is communicatively connected to the delivery device over a wired or wireless network.

24. (canceled)

25. (canceled)

26. The system of claim 22, wherein the processor, to process the input to analyze the efficacy of the agent with respect to the cognitive function of the individual, is configured to:
prior to the delivery of the agent to the individual, receive an initial input associated with the one or more parameters related to the cognitive function of the individual;
define a baseline profile for the individual based on a first set of values for the one or more parameters received in the initial input prior to the delivery of the agent to the individual; and
compare a second set of values for the one or more parameters received in the input following the delivery of the agent to the individual to the baseline profile.

27. The system of claim 26, wherein the processor, to adjust one or more of the predefined time and the predefined dosage for delivery of the agent to the individual based on the analysis, is configured to one of:
decrease one or more of the predefined time and the predefined dosage in response to a determination that the second set of values indicate a decline of the cognitive function of the individual from the baseline profile;
maintain or increase one or more of the predefined time or predefined dosage in response to a determination that the second set of values indicate an improvement of the cognitive function of the individual from the baseline profile; and
after a particular period of time, receive another input associated with the one or more parameters related to the cognitive function of the individual in response to a determination that the second set of values indicate no change of the cognitive function of the individual from the baseline profile, wherein a third set of values for the one or more parameters received in the other input after the particular period of time is compared to the baseline profile.

28.-32. (canceled)

33. The system of claim 22, wherein one or more of the reservoir of the delivery device comprises at least two cartridges and the delivery device comprises at least two reservoirs configured to hold the agent and another agent.

34. (canceled)

35. The system of claim 33, wherein the processor is further configured to:
instruct the delivery device to deliver the agent and the other agent to the individual at respective predefined times and predefined dosages;
process the input to analyze an efficacy of each of the agents with respect to the cognitive function of the individual and determine one or more interactions between the agent and the other agent; and
adjust one or more of the respective predefined times and predefined dosages for delivery of the agent and the at least one other agent to the individual based on the analysis and the determined interactions.

36. The system of claim 22, wherein the delivery device is one or more of a transdermal delivery device, an oral delivery device, a nasal delivery device, a sublingual delivery device, a buccal delivery device, an ocular delivery device, an otic delivery device, a rectal delivery device, a vaginal delivery device, an intravenous delivery device, an intramuscular delivery device, a subcutaneous delivery device, an intradermal delivery device, an intrathecal delivery device, a cutaneous delivery device, a nebulizer, and an inhaler.

37. The system of claim 36, wherein the transdermal delivery device is one of:
a wearable computing device configured to contact a portion of skin of the individual when worn by the individual;
a device configured to self-adhere to a portion of skin of the individual; and
a device integrated in a clothing item that is configured to contact a portion of skin of the individual when the clothing item is worn by the individual.

38. The system of claim 22, wherein the agent is a nootropic agent comprising a drug, a supplement, or a cognitive-enhancing substance.

39. (canceled)

40. A delivery device configured to deliver agents, the delivery device comprising:
a reservoir configured to hold an agent;
a dispenser configured to receive the agent upon release from the reservoir and dispense the agent for delivery to an individual; and
a controller coupled to the reservoir and the dispenser and configured to execute instructions received from a processor to deliver the agent to the individual at a predefined time and a predefined dosage, wherein the predefined time and the predefined dosage is adjusted based on an analysis of an efficacy of the agent with respect to a cognitive function of the individual performed by the processor.

41. (canceled)

42. (canceled)

43. The delivery device of claim 40, wherein the delivery device further comprises sensors configured to provide input associated with one or more parameters related to the cognitive function of the individual to the processor, and wherein the sensors include one or more of accelerometers, audio sensors, visual sensors, gyroscopes, and biometric sensors.

44. (canceled)

45. The delivery device of claim 40, wherein the delivery device is a transdermal delivery device that is wearable, self-adhering to a portion of skin, or integrated to a clothing item.

46.-49. (canceled)

50. The delivery device of claim 40, wherein the reservoir is refillable.

51.-53. (canceled)

54. The delivery device of claim 40, wherein the delivery device includes more than one reservoir, each reservoir configured to hold one or more of a different type of agent and a different dosage of agent.

55.-65. (canceled)
